(19) United States
(12) Patent Application Publication
(10) Pub. No.: US 2015/0174379 A1
(43) Pub. Date: Jun. 25, 2015

(54) DEVICES AND METHODS FOR THE TREATMENT OF VASCULAR DISEASE

(71) Applicants: Celso Bagaoisan, Union City, CA (US); Suresh Subraya Pai, Los Altos, CA (US)

(72) Inventors: Celso Bagaoisan, Union City, CA (US); Suresh Subraya Pai, Los Altos, CA (US)

(21) Appl. No.: 14/634,694

(22) Filed: Feb. 27, 2015

Related U.S. Application Data
(63) Continuation of application No. PCT/US13/57733, filed on Aug. 30, 2013.
(60) Provisional application No. 61/786,499, filed on Mar. 15, 2013.
(60) Provisional application No. 61/694,922, filed on Aug. 30, 2012.

(51) Int. Cl. A61M 25/10; A61M 25/09; A61M 25/00

(52) U.S. Cl. CPC A61M 25/10; A61M 25/0074; A61M 25/003; A61M 25/09; A61M 25/1011; A61M 2025/0681

(57) ABSTRACT

Apparatus and methods are provided for delivering fluid into a body lumen. The apparatus may include a catheter including proximal and distal ends, a fluid delivery lumen extending from the proximal end to a transit port on a distal portion of the catheter, and an inflatable infusion element on the distal portion such that an interior of the inflatable infusion element communicates with the fluid delivery lumen. During use, a distal end of the catheter is introduced into a lumen or conduit with the inflatable infusion element in a collapsed condition, the inflatable infusion element is expanded to an expanded condition to partially or totally occlude the lumen or conduit, and fluid is infused from the catheter into the lumen or conduit proximally relative to the inflatable infusion element while the inflatable infusion element remains in the expanded condition.

### RELATED APPLICATION DATA

This application is a continuation of co-pending International Application No. PCT/US/2013/057733, filed Aug. 30, 2013, which claims benefit of U.S. provisional applications Ser. Nos. 61/694,922, filed Aug. 30, 2012, and 61/786,499, filed Mar. 15, 2013, the entire disclosures of which are expressly incorporated by reference herein.

### FIELD OF THE INVENTION

The field of the present invention relates generally to medical devices and methods, and more specifically to catheter-based devices and methods for the treatment of diseased vasculature.

### BACKGROUND

Minimally invasive diagnostic and therapeutic interventions for the treatment of vascular disease typically use a combination of catheter and wire-based devices. In performing a typical percutaneous vascular intervention, exemplary procedural elements may include the following: obtaining a clear image of the vasculature including a vessel obstruction, lesion, or other treatment site using fluoroscopy and injection of contrast imaging agents through the lumen of a guide catheter or sheath before, during, and/or after treatment; accessing and crossing the vessel obstruction or lesion using a combination of one or more guide wires, guide catheters, support catheters, and/or sheaths; and finally treatment of the obstruction or lesion using specialized catheter-based tools (e.g., one or more angioplasty balloons, stent and stent delivery systems, atherectomy, drug delivery infusion catheters, and the like).

Regarding the first procedural element (i.e., clear imaging and visualization of the vasculature), there are multiple factors that can affect clear fluoroscopic imaging or visualization of the vasculature and the target treatment site including, but not limited to the type of device or equipment used, concentration of the contrast used, amount or flow rate of injection, vessel condition (e.g., vessel obstructions, side branches or collaterals, vessel tortousity between the injection source and treatment site, total occlusion of the treatment site, etc.), patient profile or body habitus (e.g., morbidly obese patients), distance between the injection source and the treatment site, patient clinical profile (i.e., low ejection fraction or presence of congestive heart failure), amongst other factors. Depending on specific factors that are present in a given patient, physician operators adjust the procedural approach and their choice of catheter-based devices in order to obtain the best images possible, with the disadvantage that many of these approaches and devices provide an inefficient delivery of diagnostic contrast agents or solutions, which may result in a protracted procedure time, potential safety issues, and/or increased procedural costs.

For example, iodinated, or iodine-based, contrast agents act by attenuating the signal of an X-ray passed through the body of the patient, and manifest as a darker area on the resulting radiograph. Iodinated contrast agents are available in either ionic (e.g., Hypaque 50, Isopaque 370, Hexabrix, etc.) or non-ionic (e.g., Isovue 370, Omnipaque 350, Ultravist 370, etc.) forms, and are commonly used due to their solubility and relatively benign interaction with the body. The contrast agent is introduced into the patient at a high concentration or volume to account for the dilution of contrast agent as it flows through the patient's vascular system and to compensate for losses due to flow through collateral or side-branch vessels (i.e., vessels that are not the subject of the target interventional procedure or surgery).

In a typical interventional vascular procedure, the steps include introduction of a guide wire along a guide catheter or sheath and across an obstruction using fluoroscopic guidance. During this process, a contrast agent may be injected through the lumen of the guide catheter or sheath to provide an angiographic image that identifies the position of the radiopaque distal end of the guide wire relative to the vessel. This type of contrast imaging may be repeated at any point during the procedure in order to confirm the position of a treatment device such as a balloon catheter relative to the obstruction targeted for treatment. The balloon catheter is then advanced along the guide catheter and the guide wire until the balloon is positioned across or within the obstruction. The position of the balloon is then confirmed by injecting a contrast agent through the guide catheter or sheath to provide an angiographic image prior to the surgical or endovascular treatment. The obstruction is dilated by inflation of the balloon to restore blood flow, and the final result is confirmed using conventional angiography. After treatment, the devices are removed from the patient.

In the case of peripheral vascular disease, the artery segment targeted for intervention or surgical treatment is often a considerable distance away from the source of contrast injection (e.g., the distal tip of the guide catheter or sheath). For example, peripheral vascular interventional procedures conducted on arteries that are below the knee are often imaged using contrast agent injected through a guide catheter or sheath placed antegrade in the ipsilateral common femoral artery or via a retrograde approach using the contralateral common femoral artery up and over the aortic bifurcation and into the opposite leg. The relatively long distance between the source of contrast administration and the treatment site (e.g., from the iliac to an arterial location below the knee) often necessitates the use of a higher concentration and/or a larger volume of contrast agent coupled with a longer X-ray exposure time due to the longer run off time to obtain images that are of sufficiently high quality to aid in diagnosis and treatment. The use of a higher volume of contrast agent can induce nephropathy (i.e., contrast agent-induced damage to the kidneys), which is especially relevant to a large cohort of the patient population that suffers from peripheral vascular disease (e.g., diabetics). Furthermore, the high, prolonged doses of radiation associated with these techniques are not beneficial and can be harmful to the patient and/or the treating physician and staff. In some cases, it is not possible to acquire images of high enough quality to complete the intervention or treatment.

Current techniques for obtaining clear images of the peripheral vasculature focus on moving the source of contrast administration closer to the target vessel or treatment area (i.e., use of selective angiography). For example, a relatively large and/or rigid guide catheter or sheath may be placed as close to the target vessel or treatment area as possible to minimize the dilution of contrast agent between the end of the guide catheter or sheath and the target vessel or treatment area. The balloon catheter is then advanced along the guide catheter or sheath to treat the arterial lesion and a post-treatment angiogram is taken to assess the efficacy and outcome of the treatment. While useful, this technique is restricted in that the guide catheter or sheath must have a large enough lumen to facilitate injection of a sufficient enough volume contrast agent at a desirable flow rate while the interventional equipment or treatment devices are indwelling within the lumen of the guide catheter or sheath. The larger size and/or rigidity of the guide catheter or sheath may also prohibit placement close to the smaller peripheral vasculature as desired for selective angiography.

Another technique used to conduct selective angiography includes removing the guide wire from the lumen of the balloon catheter and injecting contrast agent through the vacant guide wire lumen. The disadvantages of this technique include the loss of wire position within the vasculature, and the limitation imposed by the small size of the guide wire lumen on the volume and flow rate of contrast agent that can be injected. This limitation can compromise image quality. Furthermore, procedure time increases, as the operator must exchange the guide wire for a contrast injection manifold to perform an angiogram, then re-introduce and re-establish guide wire position within the patient after removing the manifold. This technique is also used to perform selective administration of thrombolytics or other commonly used drugs (e.g., nitroglycerin, papaverine, heparin, tPa, etc.), of which the disadvantages are similar to that described previously.

Another technique for treating peripheral vascular disease in arteries that are below the knee is to introduce the treatment device directly into an artery distal to the lesion and/or occlusion (e.g., introducing the treatment catheter via percutaneous trans-pedal access without an access sheath to treat a lesion in the calf) when lesion access from the antegrade approach is unsuccessful or prohibited. While this approach provides an alternative to the use of a larger or higher profile guide catheter or sheath used in the antegrade approach, the trans-pedal approach introduces several drawbacks. First, the contrast agent may not be easily injected through the treatment device, as described previously, inhibiting effective imaging of the treatment area. Second, as the treatment device has been inserted directly into the artery without an access-site sheath, there is no other means to obtain selective angiograms. Third, as the trans-pedal access provides a retrograde approach, injection of lower volume and concentration of contrast agent, as well as lower flow rates of injections against the incoming blood flow inhibits angiographic imaging of the target vessel and lesion. When the anatomy allows, a small diameter access-site sheath (e.g., a four (4) or five (5) French micro puncture kit) may often be used to obtain trans-pedal access in an attempt to successfully image the treatment site and limit damage to the small artery. The small luminal diameter access sheath precludes the ability to inject adequate volumes of contrast agent and to obtain the high quality angiographic images necessary for diagnosis and treatment especially when interventional tools or treatment devices are placed within it. It is also noted that in cases of total occlusion, the absence of flow through the obstruction in the treatment artery would prevent imaging of the vasculature distal of the obstruction, thus the physician is essentially navigating blind until the lesion is crossed and confirmation of the location of the treatment catheter is obtained.

Even when the angiographic images are of sufficient quality to guide the procedure and the treatment of the lesion is successful, acquiring the follow-up angiogram can be challenging. The inflatable balloon segments of catheters commonly used to treat arterial lesions are typically pleated and folded about a inner core or shaft of the catheter to minimize the cross sectional profile of the balloon and enable the balloon to more easily cross the lesion. However, the expansion and subsequent deflation of the balloon often induces the formation of substantially flat “wings” or other folded structures that are not optimally wrapped around the inner core or shaft of the catheter. These balloon wings or folded structures can impede the flow of contrast through the treated lesion while the balloon is left in place within the lesion and prevent the user from obtaining high quality images needed for accurate assessment of the efficacy of the treatment.

In current practice, when a diagnostic angiogram is required in a dialysis graft or fistula, e.g., to interrogate for venous or arterial stenosis or other defects, the interventionalist will usually first insert a sheath (typically a micropuncture sheath) into the fistula. Contrast or contrast/saline mixtures may then be injected through the side port of this indwelling sheath to opacify the venous segment of the fistula to characterize the venous segment and the presence of any defects or stenosis. Visualizing the arterial anastomosis is then accomplished by manual compression of the vein (in the arm) distal to the tip of sheath so as to enable reflux of the contrast or contrast/saline mixture towards the arterial anastomosis. The issue with this method is that the hand and/or arm of the physician or member of the cath lab's staff is directly subjected to the field of dangerous and harmful radiation while enabling visualization of the arterial anastomosis. Alternatively, if stenosis is detected in the venous segment, physicians will typically insert a percutaneous translumenal angioplasty balloon into the sheath and treat the subject area to high pressure dilation. This same balloon is also sometimes maintained in the inflated condition thereby occluding the artery while contrast is injected through the side port of the sheath to create reflux (similar to the manual compression technique) to visualize the arterial anastomosis. However, if no balloon is required due to a lack of stenosis on the venous segment, such a balloon would not be necessary and is typically not used for visualization of the arterial segment due to the high expense of such a device for only diagnostic use. Either way, the method of using reflux to visualize the arterial anastomosis is suboptimal and there is a need for improved and/or low cost devices and methods to visualize this vessel segment without directly subjecting the physician or staff to unnecessary, dangerous and harmful radiation.

Regarding the second procedural element identified above, the ability to access and cross the vessel obstruction or lesion may present a very challenging task to a physician given the vessel condition and pathology, patient profile, anatomy or body habitus and a patient's clinical profile. For example, when treating vascular obstructions in smaller vessels (e.g., below the knee), placement of a guide wire across or within the obstruction can be challenging due to relatively dense calcific, atheromatous plaque since conventional guide wires have relatively weak column strength (e.g., the guide wire often does not support the requisite amount of force for crossing the plaque without buckling or prolapsing). In order to provide supplemental support to bolster the pushability of the guide wire and enable placement of the guide wire across or within the plaque, current techniques include the use of a support catheter comprising a low profile, single or multiple lumen tube with a tapered tip. The support catheter is coaxially positioned over the guide wire to substantially augment the pushability over that of the guide wire alone and to increase the opportunity to successfully advance and position the guide wire across or within the obstruction. Once the guide wire and support catheter have crossed the obstruction, the guide wire may be exchanged with another type of guide wire (i.e., with a softer atraumatic wire), and then the support catheter may be removed from the patient while carefully maintaining the position of the guide wire across or within the obstruction. The treatment device may then be advanced along the guide wire to complete the procedure.

The use of a support catheter to bolster guide pushability and minimize buckling is further complicated by the clinical observation that the proximal edge of the plaque, or plaque cap in the case of a total occlusion, can be more dense, calcific, or fibrous than the distal portion. This presents a challenge in directing the wire across or within the plaque from the proximal or antegrade approach. In these cases, physician operators may access the obstruction via trans-pedal access using a retrograde approach. Once the guide wire crosses the obstruction, the distal tip of the guide wire is captured on the proximal side of the plaque using a snare inserted into the femoral artery via an access sheath. The snare holding the guide wire is then retracted through the femoral access sheath. The treatment device can then be loaded over the guide wire, through the femoral access sheath, and advanced distally until it is in position across or within the obstruction. In some instances, the dense nature of the plaque may continue to present a challenge to pushing the treatment device into and across the target lesion. For example, the treatment device and guide wire can still buckle or prolapse under the compressive load needed to cross the lesion.

In some instances, there can be a challenge in the placement or crossing of a treatment device (e.g., a balloon catheter) across the vessel obstruction or lesion in spite of successful crossing with a guide wire and/or support catheter. For example, the long length of the catheter shaft and/or the flexibility of the material used to construct the catheter may contribute to ineffective transmission of push forces required to cross tight vessel obstructions, resulting in prolapse, buckling and sometimes kinking of the device. In this situation, a pre-dilation of the obstruction may be performed using a smaller diameter angioplasty balloon to dilate the vessel, crack the plaque, and/or to slightly open the lumen to ease passage of the treatment device. Alternatively, de-bulking or atherectomy devices (e.g., rotablator, laser, etc.) may be used to remove or obliterate tissue to create a passage to ease placement of treatment devices. Nevertheless, these approaches can present safety issues, extend procedure time, and/or increase procedure cost.

Therefore, it would be useful to provide treatment devices and/or methods that can deliver a minimal, but sufficient amount of contrast in close proximity to a treatment location to obtain an angiogram of sufficient quality during a procedure, while minimizing the exposure of the patient and/or operator to harmful X-ray radiation and/or reducing the contrast load. Such treatment devices may also provide local and/or efficient administration of fluids such as thrombolytics or other commonly used drugs (e.g., nitroglycerin, papaverine, heparin, tPa, etc.) to eliminate the burden of exchanging and/or removing and reintroducing devices in the patient and to minimize the amount of fluid used to perform the diagnostic and/or therapeutic procedure. Moreover, such treatment devices may also have a low profile shaft to allow navigation in the distal vasculature (i.e., small diameter vessels, tortuous anatomy, etc.) and/or allow crossing of obstructions, and/or may have a balloon component that grooms or folds down to a substantially small profile close to its pre-inflated cross-sectional profile, e.g., to minimize the disruption of contrast flow past the balloon post treatment and to allow multiple passage through tight obstructions. Furthermore, such devices may combine the functionality of a support catheter with the dilating capability of an angioplasty balloon to ease guide wire and balloon placement without the need for multiple devices or exchanges. It may also be useful to provide an alternative to the current technique (i.e., manually and externally applying compression to occlude a vein or artery) with an intravascular diagnostic device in order to visualize the vessel segment without subjecting the physician or staff to unnecessary, dangerous, and harmful radiation. Finally, it may be useful to pull the treatment devices across target obstructions or lesions instead of pushing as done in conventional procedures, as the application of a tensile load will not buckle or prolapse the treatment device.

### SUMMARY

The various embodiments described herein generally provide devices, systems, and methods for the diagnosis and/or treatment of vascular disease that, among other features, may minimize the use of contrast agents, localize the delivery of contrast agents and/or other injectable fluids (i.e., liquid and gas), and/or facilitate generation of quality angiographic images with reduced operator and patient exposure to X-ray radiation. Moreover, devices, systems, and methods may also be provided for improving procedure efficiency and/or enhancing the traversal of target lesions.

In accordance with a first embodiment, a device is provided that comprises an elongate member sized for insertion into a body lumen with proximal and distal ends and multiple lumens extending there between, and a balloon arranged on a distal portion of the elongate member and in communication with at least one of the multiple lumens of the elongate member. Another lumen is provided as a guide wire lumen extending along the length of the elongate member and terminating distal to the distal tip of the balloon. A third lumen is provided as a fluid delivery lumen terminating in a port or opening that is in communication with the environment external to the device. The fluid delivery lumen may be a separate and independent lumen or lumens or may be a lumen shared with the guide wire lumen.

The proximal end of the elongate member may be joined to a manifold or other apparatus for delivering liquids and/or gases and/or other devices through the individual lumens of the elongate member. For example, a solution of diluted contrast agent may be injected through the lumen in communication with the balloon to inflate the balloon. The balloon may be deflated by application of negative (or vacuum) pressure to the same lumen. A guide wire or other device may be inserted into the guide wire lumen to provide a rail or method for directing the device to the desired location within a patient. A solution of contrast agent may be injected through the fluid delivery lumen to provide a means of obtaining an angiogram of the anatomy in proximity to the device.

An exit port or opening is provided at the distal end of the fluid delivery lumen and may be located proximal to the proximal end of the balloon, and may comprise a single opening, or multiple openings distributed about the length and/or circumference of the elongate member. Alternatively, the exit port or opening of the fluid delivery lumen may be located distal to the distal end of the balloon, or the exit port or opening may have multiple openings located both proximal of and distal to the balloon.

The multiple lumens of the elongate member may maintain the same diameter from their proximal ends to their distal ends, or they may vary in diameter and location along the length of the elongate member. Yet another alternative is to provide a cover or shield over the exit port or opening such as a tubular member, an expandable member (such as a balloon) and the like. In this configuration, the exit port or opening may not directly communicate with the environment external to the device, such as a blood vessel or lumen, and only functions as a transit port for fluid infused through the fluid delivery lumen that fills the internal space of the said cover or shield. The cover or shield may be made from a non-compliant or compliant material, which may be occlusive or non-occlusive to the blood vessel or lumen. The cover or shield may also comprise a micro exit hole or port or opening that aims the infused fluid in the retrograde or antegrade direction.

For example, a cover or shield utilizing an expandable member (e.g., an inflatable balloon) may comprise an exit port or opening located at the distal side of the cover or shield whereby the fluid infused through the exit port or opening is projected in the distal direction. On the other hand, an exit port or opening located at the proximal side of the cover or shield may project the infused fluid in the proximal direction. The trajectory or path of the fluid jet coming out of the exit port or opening may be aimed or projected along or at some angle relative to the longitudinal axis or path of the blood vessel or lumen. A cover or shield that is occlusive to the blood vessel or lumen may provide a benefit of blocking flow of blood during fluid injection to allow a more efficient, effective, and/or concentrated mode of delivery since the injected fluid is isolated from flowing blood, preventing further dilution due to mixing of fluid with flowing blood. This may be particularly useful when delivering drug or other therapeutic agents where a need to maintain the delivered concentration is useful.

In the case of a diagnostic procedure, blocking the blood flow may allow the contrast agent or solution to be delivered in the retrograde direction, which may be particularly useful when performing diagnostic imaging of, for example, an arterial anastomosis during a dialysis graft intervention. Another potential benefit for providing a cover or shield that aims the infused fluid along the length of the blood vessel or lumen is that the fluid jet exiting the hole or opening (which may be delivered using a power injector) may be projected substantially along the longitudinal axis or path of the vessel wall such that by the time the fluid jet contacts the wall of the vessel or lumen, the energy or force of fluid jet is significantly reduced or diminished, thus potentially eliminating the risk of vessel wall dissection, damage, or trauma caused by a powerful fluid jet.

In accordance with yet another embodiment, a diagnostic or treatment device may be provided including an elongate member comprising a single lumen. The alternative device configuration disclosed herein may be similar or otherwise related to the devices disclosed in co-pending U.S. Patent Application No. 61/694,922, incorporated by reference herein. Similar to all of the aforementioned device configurations, a single or multiple exit port or opening may be provided as a pass through for fluid between the lumen of the elongate member and the space external to the lumen of the device. This exit port or opening may be configured to not directly communicate with the environment external to the device when a compliant or non-compliant tubular member, and/or an expandable member such as a balloon, is provided as a cover or shield.

In a configuration where an expandable member such as a balloon is used as a cover or shield over the exit port or opening, the lumen of the elongate member may serve as both a fluid delivery lumen and an inflation lumen, thus delivery or injection of fluid may simultaneously inflate the expandable member and deliver fluid in the vasculature external to the device. In the configuration where a compliant tubular member is used as a cover or a shield, the compliant tubular member may serve as a mechanical seal at or over the exit port or opening prior to injecting or delivering fluid and may prevent any fluid external to the device such as blood from entering the device.

Upon injection or delivery of fluid, the cover or shield may expand due to the increase in fluid pressure generated internally, thus allowing delivery of fluid to the space external to the device such as a vessel or artery. As soon as the fluid injection or delivery is completed, the compliant cover or shield contracts or recoils back to mechanically re-seal the exit port or opening. When a negative pressure is applied to the device (e.g., using a syringe attached to the proximal end of single lumen of the device to create an internal vacuum), the mechanical seal provided by the compliant cover or shield may prevent external fluid such as air or blood from being suctioned into the device. In the case where the device internal pressure is left at neutral (i.e., not pressurized) and the external pressure (such as blood pressure) is greater than the internal pressure, the compliant cover or shield may prevent such fluid from entering the device.

The compliant tubular member may be attached directly to the proximal end of the balloon thereby acting as a proximal seal for the balloon to the shaft while simultaneously covering the exit port or opening at the attachment point between the proximal end of the balloon and the elongate member. Alternatively, the proximal end of the balloon may be permanently attached to the elongate member and the elongate member may include an exit port proximal to the inflatable member of the balloon covered by a compliant tubular member. The compliant tubular member may be attached directly to the elongate member or over the attachment between the balloon and elongate member. In a further alternative embodiment, the design may have a configuration such that the exit port or opening is located distal of the expandable member wherein fluid infusion is directed distally.

Optionally, the device may comprise features for regrooming or refolding an expandable member, such as a balloon, into a low profile about the elongate member. The device may include a balloon and an inner elongate member attached to the distal end of the elongate member. The distal end of the expandable member is attached to the distal end of the inner elongate member. The expandable member is configured relative to the inner elongate member such that the collapsed expandable member approximates the surface of the inner elongate member (e.g., such that the balloon in a collapsed state would lie in contact and extend along a section of the inner elongate member). At the same time, the expandable member may be rotated about the longitudinal axis such that the distal end attached to the inner elongate member is at some radial offset relative to the proximal end of the expandable member (e.g., the distal end of the expandable member is rotated about its longitudinal axis by 90, 180, 270, or 360 degrees, or to any desired angle). This configuration may provide at least one spiral pleat along the length of the collapsed expandable member. When the expandable member is expanded, it is forced to rotate about the longitudinal axis in the opposite direction and the at least one spiral pleat may untwist as it expands. This process may apply torque to the inner elongate member such that its proximal end is at some offset rotational angle relative to its distal end. The application of torque to the inner elongate member may store potential energy along the length of the inner elongate member. Upon collapse of the expandable member, the potential energy stored in the inner elongate member is released as it rotates back towards its original position. The expandable member then forms at least one substantially spiral pleat along the length of the balloon as it returns to the collapsed configuration.

One method of using devices described herein is to treat peripheral vascular disease located below the knee. In this method, access to the vasculature is obtained using the Seldinger or other known technique with either an antegrade insertion into the ipsilateral femoral artery or a retrograde insertion into a contralateral femoral artery up and over the aortic bifurcation and into the opposite leg. A guide wire is then used to advance a sheath or guide catheter into a position as near the lesion as possible (within the limits of vessel size, tortousity, sheath rigidity, body habitus and sheath dimension). A device, such as the first embodiment described above, may be advanced along the guide wire and along the sheath or guide catheter to a position that is closer to the lesion than the distal end of the sheath or guide catheter.

A select volume of contrast agent solution is introduced into the manifold and delivered through the fluid delivery lumen of the treatment device. The term “select volume of contrast agent solution,” as used herein, refers to the minimized amount of contrast solution required to obtain high quality images by exploiting the smaller fluid delivery lumen and exit ports, as well as the ability to position the low profile device closer to the target lesion or vessel. The contrast is delivered to the vasculature immediately about the exit port or ports of the treatment device enabling visualization and recording of an angiogram of the lesion and surrounding vasculature prior to treatment.

The guide wire is then advanced across the lesion followed by the distal end of the device to place the balloon in a position to treat the lesion. Inflation media, such as a solution of contrast agent and saline and/or another suitable liquid, is introduced into the lumen that communicates with balloon to expand the balloon and treat the lesion. The balloon is deflated and refolds substantially to its original configuration about the elongate member of the device, enabling improved passage or flow of contrast agent solution by the balloon. At this point, a select volume of contrast agent solution is again introduced into the manifold and delivered through the fluid delivery lumen to the vasculature immediately about the exit port or ports of the device and a post-treatment radiograph of the vasculature about the treated lesion is viewed and recorded. The device, guide wire, and sheath or guide catheter are then removed from the patient.

Another method of using a device, such as the first embodiment described above, to treat, for example, a below-the-knee arterial lesion comprises gaining access to the vasculature of the patient via percutaneous, trans-pedal access. In this method, the device of the first embodiment comprises exit ports in the fluid delivery lumen that are located proximal and/or distal to the balloon. Access to the vasculature is obtained via the Seldinger or other known technique, leaving a guide wire placed in the access artery. The device of the first embodiment is advanced over the guide wire and into the patient. At this point, the operator may use the manifold to inject contrast through the fluid delivery lumen to obtain an angiogram of the anatomy distal to the lesion, and use the one or more images obtained to place the guide wire across the lesion.

The balloon segment of the device is advanced over the guide wire and across the lesion. The operator may then optionally use the manifold to inject contrast through the fluid delivery lumen and obtain an angiogram of the anatomy proximal and/or distal to the lesion, as the distal exit port of the device is located proximal to the lesion and the proximal exit port of the device is located distal to the lesion. As defined previously, a select volume of contrast solution or other suitable liquid is introduced into the lumen that communicates with balloon to expand the balloon and treat the lesion. The balloon is deflated and refolds to substantially its original configuration about the elongate member of the device. At this point, a select volume of contrast solution is again introduced into the manifold and delivered through the fluid delivery lumen to the vasculature immediately about the exit port or ports of the device and a post-treatment angiogram of the vasculature about the treated lesion is taken. The device and guide wire are then removed from the patient.

In accordance with another embodiment, a device is provided that comprises an elongate member sized for insertion into a body lumen with proximal and distal ends and multiple lumens extending there between, a balloon arranged on the distal portion of the elongate member and in communication with at least one of the multiple lumens of the elongate member. Another lumen of the elongate member may extend through the balloon, terminate distal to the distal tip of the balloon, and be sized to receive a standard guide wire.

The portion of the elongate member extending distal of the distal tip of the balloon may comprise a guide wire support segment. The guide wire support segment may be coaxially arranged about the guide wire lumen such that when used in conjunction with a guide wire, the overall pushability of the guide wire is amplified to enhance crossing of significantly tight obstructions or lesions. The guide wire support segment may optionally comprise a tapered tip on the distal end to enable easier insertion and passage through constricted body lumens. A portion of the guide wire support member may comprise a hydrophilic coating and/or angiographically visible markers to aid in characterizing lesion length and/or other characteristics of the lesion.

The lumen may comprise a variable diameter along the length of the elongate member, e.g., with a larger diameter at the proximal portion of the elongate member transitioning to a smaller diameter towards the distal portion of the elongate member. The diameter of the proximal portion of the lumen may be sized to accommodate a standard guide wire and maintain enough space to permit flow of a contrast solution through the lumen and about the guide wire when the guide wire is positioned in the larger diameter portion of the lumen. The diameter of the smaller portion of the lumen may be sized to closely approximate the outer diameter of a standard guide wire and reduce or limit flow of a contrast solution through the lumen and about the guide wire when the guide wire is positioned in the smaller portion of the lumen. The transition from the larger diameter to the smaller diameter may be located proximal to the balloon, between the proximal and distal ends of the balloon, or distal to the distal end of the balloon. The geometry of the transition may be symmetric or asymmetric about the centerline of the lumen. Furthermore, the transition may be reinforced to increase strength or prevent deformation using materials including, but not limited to high-strength plastics, metals, braids, composites thereof, and the like. One or more exit ports may be located along the length and/or circumference of the elongate member that enable communication between the environment external to the elongate member and the guide wire lumen. The transition section of the guide wire lumen may be located relative to the exit ports in such a manner that the insertion of a guide wire in the guide wire lumen preferentially directs flow of a select volume of contrast solution, as defined previously, out of a desired exit port or ports. For example, the transition section may be located between the proximal end of the balloon and an exit port located proximal to the transition section, such that the exit port is located in the portion of the lumen with the larger diameter. The insertion of a guide wire through the guide wire lumen creates an area of increased resistance to flow at the portion of the lumen with the smaller diameter, directing the flow of contrast out of the exit port. In another example, the transition section may be located distal to an exit port that is in turn distal to the distal end of the balloon. The guide wire lumen in this example may further comprise a second exit port located proximal to the proximal end of the balloon, thus placing both exit ports in the area of larger diameter. The insertion of a guide wire through the guide wire lumen creates an area of increased resistance to flow at the portion of the lumen with the smaller diameter, directing the flow of contrast out of the exit ports. The guide wire lumen may be sized to optionally accept more than one guide wire to improve the pushability of the device.

Alternatively, the fluid delivery lumen may maintain a constant diameter of sufficient dimension to allow a select volume of contrast as defined previously to flow through the fluid delivery lumen when a guide wire is resident within the fluid delivery lumen. In this case, contrast may be delivered out of the exit ports and out of the distal tip of the fluid delivery lumen. It should be clear to one of skill in the art that the contrast delivery aspect of the device may be eliminated, resulting in a device that comprises a guide wire lumen with one or more transitions.

The proximal end of the elongate member may be joined to a manifold or other apparatus for the delivery of liquids and/or gases and/or other devices through the individual lumens of the elongate member. For example, inflation media comprising diluted contrast solution may be injected through the lumen in communication with the balloon to inflate the balloon. The balloon may be deflated by application of a negative pressure (i.e., vacuum) to the same lumen. The device may include one or more features for regrooming or refolding the balloon about the elongate member during deflation, e.g., to substantially reduce the balloon's cross sectional profile and/or enable easier passage of contrast by the balloon, as previously discussed.

In accordance with yet another embodiment, a reinforced guide wire is provided that includes a removable jacket. The removable jacket is an elongate member with proximal and distal ends and a lumen therethrough. The guide wire is disposed within the lumen of the removable jacket and secured through a reversible locking element that interacts with the removable jacket. The reversible locking element may include one or more of a Touhy-Borst valve resident on the removable jacket, a key and lock mechanism wherein the key feature may be located on the guide wire and the lock feature may be located on the removable jacket or vice versa, a ratchet or toothed mechanism resident on the guide wire, the removable jacket, or both, a tap/thread mechanism wherein the tap is located on the removable jacket and the thread is located on the guide wire or vice versa, a living hinge/detent system, and the like. The distal end of the removable jacket may taper inward to a position flush with the outer diameter of the guide wire, or may be configured as an abrupt transition comprising a blunt end that does not smoothly mate with the guide wire. For example, the distal end of the removable jacket may be a convex or concave taper, a stepped transition, a linear transition of steep or shallow angle, and the like. The lumen of the guide wire may be sized to closely approximate the outer diameter of common guide wires (e.g., having an inner diameter of at least about 0.25 mm (0.010)″, 0.35 mm (0.014″), 0.45 mm (0.018″), 0.88 mm (0.035″).

In an exemplary method of use, a balloon catheter may be loaded onto the reinforced guide wire and inserted into the patient. The removable jacket may provide support to the guide wire and balloon catheter during the advancement of the guide wire and the balloon catheter, e.g., to and across a lesion. The removable jacket may then be decoupled from the guide wire and removed off of the guide wire and out of the guide wire lumen of the balloon catheter to allow the flow of a contrast media around the guide wire and out any of the exit ports resident on the elongate member of the balloon catheter.

One method of using such a balloon catheter and reinforced guide wire may also use a catheter (e.g., guide catheter or sheath), and a snare to treat an obstruction or arterial lesion located below the knee. One advantage of this technique over current conventional methods of access is that the balloon catheter may be pulled from the distal end to cross the lesion as opposed to being pushed from the proximal end. Pulling the catheter from the distal end may provide better leverage and crossing force instead of pushing the proximal end as done in conventional procedures, since the application of a tensile load will not buckle or prolapse or otherwise compress the treatment device. The balloon catheter employed in this method may include a guide wire lumen that tapers from a larger proximal inner diameter to a smaller distal inner diameter at a point close to the distal end of the balloon catheter.

The method comprises obtaining access to the vasculature of the patient using a guide catheter or sheath at a point proximal to the lesion (e.g., an antegrade ipsilateral common femoral artery puncture, a retrograde contralateral common femoral artery puncture up and over the aortic bifurcation and into the opposite leg, etc.), and advancing the tip of the guide catheter or sheath as close as possible to the arterial lesion. The distal vasculature may be accessed using a percutaneous trans-pedal technique and a suitably sized guide wire is placed intravascularly. A micro puncture sheath may be used at this access point if desired.

The balloon catheter is then loaded onto the guide wire, followed by the removable jacket. The removable jacket is inserted along the guide wire lumen of the balloon catheter and advanced until the distal end taper of the removable jacket contacts the guide wire lumen transition taper in the distal segment of the balloon catheter. Alternatively, the removable jacket may be pre-assembled and resident inside the guide wire lumen of the balloon catheter prior to loading the balloon catheter (and resident removable jacket) onto the guide wire. The guide wire, removable jacket, and balloon catheter are advanced and positioned at or near the distal edge of the obstruction or arterial lesion.

The guide wire is subsequently advanced proximally across the lesion and into the artery proximal to the lesion, with the balloon catheter and removable jacket providing support to the guide wire. A snare is advanced through the antegrade (i.e., proximal) guide catheter or sheath and the distal end of the guide wire is captured and withdrawn extracorporeally (i.e., external to the patient) through the lumen of the guide catheter or sheath. The guide wire portion external to the patient is then released from the snare. At this point, the removable jacket is coupled to the guide wire.

Using fluoroscopic guidance, the guide wire is retracted, applying a tensile force and in doing so, engages the removable jacket against the guide wire lumen transition taper in the distal segment of the balloon catheter. The continued application of a tensile force to the guide wire advances the expandable member of the balloon catheter across the lesion. The balloon catheter may include radiopaque marker bands that may aid in positioning. The expandable member is then expanded to treat the lesion and subsequently deflated. At any point during the procedure, the removable jacket may be decoupled from the guide wire and retracted proximally out of the balloon catheter to provide space in the lumen for contrast injection, and an angiogram may be taken to ascertain the location of the distal tip of the guide wire and the balloon catheter in relation to the arterial lesion. Furthermore, the refolding or regrooming embodiments described previously may be integrated into the device of this embodiment to provide easier passage of contrast past the deflated balloon.

It should be clear to one of skill in the art that this method may be applicable to any medical procedure where the distal end of a guide wire may be captured and retracted with a tensile force to enable the simultaneous positioning of a treatment device. For example, the pulling technique may be used to advance an atherectomy catheter within or across an arterial lesion. The method may be extended to include actions such as sequentially or simultaneously pushing and pulling a catheter-based medical device to place it in a desired position. This method provides a method to cross lesions that may be untreatable using traditional procedures (e.g., pushing the proximal end of catheter).

In accordance with still another embodiment, the use of a pulling technique may be facilitated using a locking guide wire that includes an elongate member with proximal and distal ends, and an expandable section located at the proximal end, distal end, or between the proximal and distal ends with a cross-sectional area that may be adjusted by the user. In the baseline state, the expandable section is about the same outer diameter and/or cross-sectional area as the remainder of the elongate member. When placed in the active state, the diameter and/or cross-sectional area of the expandable section increases to a magnitude larger than that of the elongate member. The nominal diameter of the guide wire in the baseline state may include, but is not limited to, outer dimensions of not more than about 0.25 mm (0.010)″, 0.35 mm (0.014″), 0.45 mm (0.018″), 0.88 mm (0.035″), and the like. The expandable section may comprise inflatable balloons, flexible sheaths or tubes that wrinkle or bunch when their proximal and distal ends are brought closer to each other, braids or woven structures that expand when activated, struts, arms, beams, projections, or other features that radiate away from the elongate member when activated. The expandable section may be able to reversibly transition between the baseline and active states, or it may be prevented from returning to the baseline state once activated.

One method of using this embodiment is to insert the locking guide wire inside a catheter-based medical device (e.g., a balloon catheter, atherectomy catheter, etc.) in the baseline state, advance the guide wire such that the distal tip of the guide wire is distal to the distal end of the medical device and the expandable section of the guide wire is within the lumen of the medical device, and place the expandable section of the guide wire in the active state. The increased diameter and/or cross-section of the expandable section interferes with the guide wire lumen of the medical device and engages the two devices. The medical device may then be advanced in the distal direction by pulling on the distal end of the locking guide wire with the expandable element, as previously described. Once the medical device is placed in the desired position (e.g., across an arterial lesion), the expandable section may optionally be returned to the baseline state, disengaging the locking guide wire from the medical device. It should be clear to one of skill in the art that the location of the expandable element of the locking guide wire with respect to the medical device may be proximal to the proximal end of the medical device, between the proximal and distal ends of the medical device, or distal to the distal end of the medical device. Furthermore, while the exemplary method of use describes an interference between the activated expandable segment of the locking guide wire and the lumen of the medical device, any method for reversibly or irreversibly engaging the guide wire and the medical device is contemplated (e.g., reciprocal locking features on the guide wire and medical device, restricted segments of the medical device lumen, internal or external detents, flanges, and the like).

Yet another embodiment that facilitates the use of a pulling technique is a catheter-based medical device (e.g., balloon catheter, atherectomy catheter, etc.) that comprises one or more features to engage a guide wire such that the catheter and guide wire do not translate relative to each other. Optionally, the feature(s) may allow the catheter and guide wire to rotate freely with respect to each other while being axially coupled. These feature(s) may include, but are not limited to, one or more Touhy-Borst valves, set screws, living hinges, iris clamps, expandable elements such as internal bladders, flexible segments that wrinkle or bunch under compressive loads, braided segments that decrease in inner diameter under tensile load, and the like.

One method of using this embodiment is to insert a guide wire inside the catheter while the locking feature(s) are in the baseline state, advance the guide wire such that the distal tip of the guide wire is distal to the distal end of the catheter, and activate the locking feature(s) of the catheter to engage the two devices. The catheter may then be advanced in the distal direction by pulling on the distal end of the guide wire as previously described. Once the catheter is placed in the desired position (e.g., across an arterial lesion), the locking feature(s) may optionally be returned to the baseline state, disengaging the catheter and guide wire.

In accordance with another embodiment, a support catheter is provided comprising a first elongate member with proximal and distal ends and at least one lumen therethrough. The lumen may further comprise a proximal and distal section of differing annular cross-section. For example, the proximal and distal sections may comprise circular annular cross sections having different diameters. In this example, the proximal section may have a larger cross-section than the distal section, or vice versa. In another example, the proximal section may comprise an annular cross-section that is square in cross section and the distal section may comprise an annular cross-section that is of the same area and diamond shaped in cross section (i.e., the square cross-section of the proximal section rotated about the longitudinal axis of the support catheter by about forty five degrees).

It should be clear to one of skill in the art that the differences in annular cross-section between the proximal and distal section may comprise any geometrical configuration or pair of configurations. The transition between the proximal and distal sections may be abrupt (e.g., including a step change in diameter for circular cross sections) or gradual (e.g., including a taper between two differing diameters for circular cross sections).

The elongate member may optionally comprise an infusion lumen extending from the proximal end to the distal end of the elongate member. The infusion lumen may further comprise one or more exit ports to provide a path between the infusion lumen and the environment external to the support catheter. The exit ports may be located at any radial and/or longitudinal position along the length of the infusion lumen. The proximal end of the infusion lumen may be connected to a port that enables the infusion of liquids through the lumen and out the distal end and/or exit ports of the lumen. The liquids may comprise materials including, but not limited to contrast, saline, solutions of contrast and saline, solutions of therapeutic agents, combinations thereof, and the like.

The support catheter may further comprise a second elongate member coaxially disposed within the first elongate member; the second elongate member comprising a proximal end, a distal end, and a lumen extending therethrough. The proximal end of the second elongate member may further be constrained to maintain a position at or proximal to the distal end of the first elongate member. The second elongate member may translate freely with respect to the first elongate member, and may optionally rotate freely with respect to the first elongate member. The first elongate member may be of a length such that the second elongate member may be positioned entirely within the first elongate member (i.e., the distal end of the second elongate member is aligned with the distal end of the first elongate member).

The first and second elongate members may have one or more features to control the position of the distal end of the second elongate member with respect to the distal end of the first elongate member. Such feature(s) may include, but are not limited to detents, living hinges, key and keyhole designs, tapped holes, screws, set screws, valves, combinations thereof, and the like.

For example, the first elongate member may further comprise a window extending some distance along the length of the elongate member and a threaded hole. The second elongate member may further comprise a post extending radially outwardly away from the longitudinal axis of the support catheter and through the window of the first elongate member. The support catheter may further comprise a screw inserted into the tapped hole of the first elongate member. The position of the distal end of the second elongate member relative to the distal end of the first elongate member may be adjusted by advancing the post in the proximal or distal directions. When the desired position is located, the screw may be tightened to press against the second elongate member and fix the position of the second elongate member relative to the first elongate member.

The first elongate member may further comprise markings that convey information about the location of the distal end of the second elongate member relative to the distal end of the first elongate member, e.g., using methods known in the art including, but not limited to etching, labeling, pad printing, molding, machining, combinations thereof, and the like. It should be evident to one of ordinary skill in the art that such markings may also convey information about the rotational position of the second elongate member with respect to the first elongate member.

In accordance with still another embodiment, a support catheter is provided that includes a first elongate member with proximal and distal ends and a lumen extending there through. The support catheter further comprises an expandable elongate member with proximal and distal ends and a lumen extending there through. The proximal end of the expandable elongate member is joined to the distal end of the first elongate member such that the lumen of the first elongate member is in communication with the lumen of the expandable elongate member. The support catheter further comprises a distal segment with proximal and distal ends and a lumen extending there through. The proximal end of the distal segment is joined to the distal end of the expandable elongate member such that lumen of the expandable elongate member is in communication with the lumen of the distal segment.

The expandable elongate member may comprise structures known in the art including, but not limited to, flexible membranes or tubing, metallic or polymeric braids, stents, combinations thereof, and the like. Furthermore, the expandable elongate member may exist in a baseline or active state. The annular diameter of the expandable elongate member in the baseline state is such that a guide wire may freely pass through the lumen of the expandable elongate member. When activated, the external diameter of the expandable elongate member increases. The expandable elongate member may reversibly transition between the active and baseline states.

The distal segment may be non-expandable and/or rigid, and maintain its annular and external diameters irrespective of the state of the expandable elongate member. The annular diameter of the distal segment is such that a guide wire may freely pass through the lumen of the distal segment. The first elongate member may further comprise one or more exit ports that communicate between the lumen of the distal segment and the environment external to the support catheter. The first elongate member may also include one or more features for selectively blocking or plugging the one or more exit ports to minimize or prevent fluid injected into the lumen of the first elongate member from flowing out of the one or more exit ports. Furthermore, the distal segment may comprise a valve or a seal such as an o-ring, duckbill valve, and the like that provides a seal between the annular wall of the distal segment with or without the presence of a guide wire and enables the generation and/or maintenance of a relatively high internal pressure within the support catheter.

For example, the presence of a guide wire within the lumen of the support catheter may reduce or impede flow of fluid out of the distal end of the guide wire. The internal pressure created by the introduction of fluid into the lumen of the support catheter may then expand the flexible membrane. Reducing the pressure in the lumen of the support catheter allows the flexible membrane of the expandable elongate member to return to its baseline annular and external diameters.

The support catheter with expandable elongate member may be used to dilate a lesion. In a typical procedure, the guide wire is exchanged with another guide wire, such as a guide wire having a more atraumatic tip (e.g., a soft tip), while the support catheter is in position across the lesion. The support catheter may then be removed and the treatment catheter inserted over the guide wire and advanced to cross the lesion. There are situations, as in the case of a totally occluding lesion, where the treatment catheter is unable to cross the totally occluding lesion after successful crossing of the guide wire.

In this case, the treatment catheter may be replaced with another treatment device, for example, a smaller diameter balloon catheter having a smaller profile, which is then used to cross the lesion and pre-dilate the lesion to create a larger lumen opening. The treatment catheter is then reintroduced and advanced over the guide wire and across the lesion and the treatment is performed.

The use of additional equipment or treatment devices may be eliminated by using a support catheter. In this procedure, a guide wire is introduced and placed at or near the lesion followed by the support catheter with its expandable elongate member in the baseline state. The guide wire is advanced to cross the lesion followed by advancement of the support catheter across the lesion such that the expandable elongate member is positioned within or across the lesion. Fluid is injected into the lumen of the support catheter, increasing the internal pressure in the support catheter, stretching out the expandable elongate member and effectively dilating the lesion to increase the lumen and allow subsequent passage of a treatment catheter (e.g., a balloon angioplasty catheter, dilatation catheter, artherectomy catheter, etc.). The internal pressure in the support catheter is then released, causing the expandable elongate member to collapse towards its initial configuration, e.g., returning to its baseline annular and external diameters. Additional embodiments may include a treatment catheter (including those disclosed herein) and the expandable elongate member described herein.

In cases where a typical support catheter is unable to cross the lesion after successful crossing of the guide wire, other devices and/or methods may be used such as de-bulking or atherectomy devices (e.g., rotablator, laser, etc.) to remove or obliterate tissue to create a passage to ease placement of treatment devices. The use of these alternative devices and/or methods may be eliminated or avoided by providing a support catheter in this embodiment in combination with a guide wire that incorporate design features to lock or engage these two devices together and/or using the method described previously, e.g., where the devices move in unison as these devices are pulled from their distal side or distal end in order to cross the lesion.

One of the advantages of the devices and methods described herein is the ability to perform selective angiography through the treatment catheter itself resulting in use of less contrast, thereby reducing the risk of nephropathy and decreasing physician and patient exposure to harmful radiation. The capability of the treatment catheter to conduct selective angiography may eliminate the need for a larger profile angiographic catheter such a guide catheter or sheath. For example, in an intervention conducted using an up-and-over procedure (where the guide catheter or sheath is placed over the aortic bifurcation), the distal end of the guide catheter or sheath may be placed in the iliac artery, reducing potential vessel trauma and cumbersome procedures associated with advancing the distal tip of the guide catheter or sheath near or at the target treatment area (as is customarily done). The lower profiles of the treatment devices herein may enable positioning of the treatment devices closer to the target treatment area, reducing contrast agent loss to collateral vasculature due to improved vessel selection.

Another advantage of a lower profile treatment device is a reduction in diameter of the sheath required to obtain access to the patient's vasculature, reducing the potential for complications related to access site closure and potentially eliminating the need for access site closure devices. This advantage is particularly apt during procedures that involve trans-pedal access. Furthermore, the position of the guide wire in the vasculature may be maintained during contrast injection, reducing procedure time as exchange maneuvers between the guide wire and contrast injection manifold are eliminated.

In embodiments described herein that comprise one or more exit ports located distal to the expandable member, the physician or other operator may obtain high quality angiographic images of the vasculature about and distal to the obstruction. For example, during the treatment of long, diffuse lesions, flow of contrast from a guide catheter or sheath positioned proximal to the lesion (per conventional technique) may be obstructed due to a lack space between the lesion and the balloon. Using a treatment device, such as those described herein, may enable delivery of contrast from a point distal to the balloon (i.e., not obstructed by balloon) and/or improve the characterization of the lesion and surrounding vasculature. Furthermore, local administration of fluids such as thrombolytics or other commonly used drugs (e.g., nitroglycerin, papaverine, heparin, tPa, etc.) may be achieved more effectively by using the fluid delivery lumen of the embodiments described herein.

In embodiments that comprise one or more exits ports located proximal of and distal to the expandable member, blood may perfuse through the proximal and distal exit ports whereas conventional treatment devices may occlude blood flow. In embodiments that comprise a folding or regrooming expandable member, the reduced profile of the regroomed or folded expandable member may allow for improved contrast flow past the expandable member and subsequent improvement of the resulting angiographic images. In embodiments that comprise a segment of the elongate member distal to the expandable member constructed to provide enhanced guide wire support, the need for a separate support catheter may be eliminated, reducing procedure time and expense.

These and other objects, advantages, and features of the invention will become apparent to those persons skilled in the art upon reading the details of the disclosure as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1A depicts a plan view and cross-sectional views (A-A, B-B) of a first exemplary embodiment of a catheter including multiple independent lumens for a guide wire, inflation of a balloon, and infusion of fluids.

FIG. 1B depicts a plan view and cross-sectional views (A′-A′, B′-B′) of the catheter of FIG. 1A with a guide wire placed in the guide wire lumen.

FIG. 2A depicts a plan view and cross-sectional views (A-A, B-B) of a second exemplary embodiment of a catheter comprising multiple independent lumens wherein the guide wire lumen is common with the lumen used for infusion of fluids.

FIG. 2B depicts a plan view and cross-sectional views (A′-A′, B′-B′) of the catheter of FIG. 2A with a guide wire placed in the guide wire lumen.

FIG. 3A depicts a plan view and cross-sectional views (A-A, B-B) of an alternative embodiment of the catheter of FIG. 2A wherein the infusion ports are isolated to inflatable infusion elements that provide directionality to the infusion.

FIG. 3B depicts a plan view and cross-sectional views (A′-A′, B′-B′) of the catheter of FIG. 3B with a guide wire placed in the guide wire lumen.

FIGS. 3C-3G depict plan views and cross sectional views (A-A) of alternative embodiments of catheters that include an inflatable infusion element wherein the infusion port is directed proximally towards the hub of the catheter.

FIGS. 4A-4E depict plan views of an exemplary embodiment of a reinforced guide wire and several iterations of a removable jacket taper that may be secured over the guide wire.

FIG. 5A depicts a plan view of an exemplary embodiment of a reinforced guide wire with removable jacket inserted in an exemplary embodiment of a balloon catheter.

FIG. 5B depicts a plan view of the reinforced guide wire inserted the balloon catheter of FIG. 5A with a removable jacket retracted proximally off the fluid delivery/guide wire lumen.

FIG. 6 depicts a flowchart of the steps involved in an exemplary method for treating a peripheral arterial lesion.

FIGS. 7A-7F depict cross-sections of an artery showing schematics of the steps involved in an exemplary method for treating a peripheral arterial lesion.

FIGS. 8A-8F depict cross-sections of an artery showing schematics of the steps involved in an alternative method for treating a peripheral arterial lesion.

FIG. 9 depicts a plan view of an exemplary embodiment of a support catheter.

FIGS. 10A-10B depict plan views of another exemplary embodiment of a support catheter including a telescoping segment.

FIGS. 11A-11C depict plan views of yet another exemplary embodiment of a support catheter including an expandable segment.

FIG. 12 depicts a flowchart of the steps involved in an exemplary method for dilating an arterial lesion using a support catheter including an expandable segment.

### DETAILED DESCRIPTION OF THE EXEMPLARY EMBODIMENTS

Before the present invention is described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit, is included unless the context clearly dictates otherwise, between the upper and lower limits of that range is also specifically disclosed. Each smaller range between any stated value or intervening value in a stated range and any other stated or intervening value in that stated range is encompassed within the ranges recited. The upper and lower limits of these smaller ranges may independently be included or excluded in the range, and each range where either, neither or both limits are included in the smaller ranges is also encompassed within the ranges recited, subject to any specifically excluded limit in the stated range. Where the stated range includes one or both of the limits, ranges excluding either or both of those included limits are also included in the ranges recited.

Unless defined otherwise, all technical and scientific terms used herein have the same meaning as commonly understood by one of ordinary skill in the art. Although any methods and materials similar or equivalent to those described herein can be used in the practice or testing of the present invention, some potential and exemplary methods and materials are now described. All publications mentioned herein are incorporated herein by reference to disclose and describe the methods and/or materials in connection with which the publications are cited. It is understood that the present disclosure supersedes any disclosure of any incorporated publication to the extent there is a contradiction.

It must be noted that as used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present application is not entitled to antedate such publication by virtue of prior invention. Further, the dates of publication provided may be different from the actual publication dates, which may need to be independently confirmed.

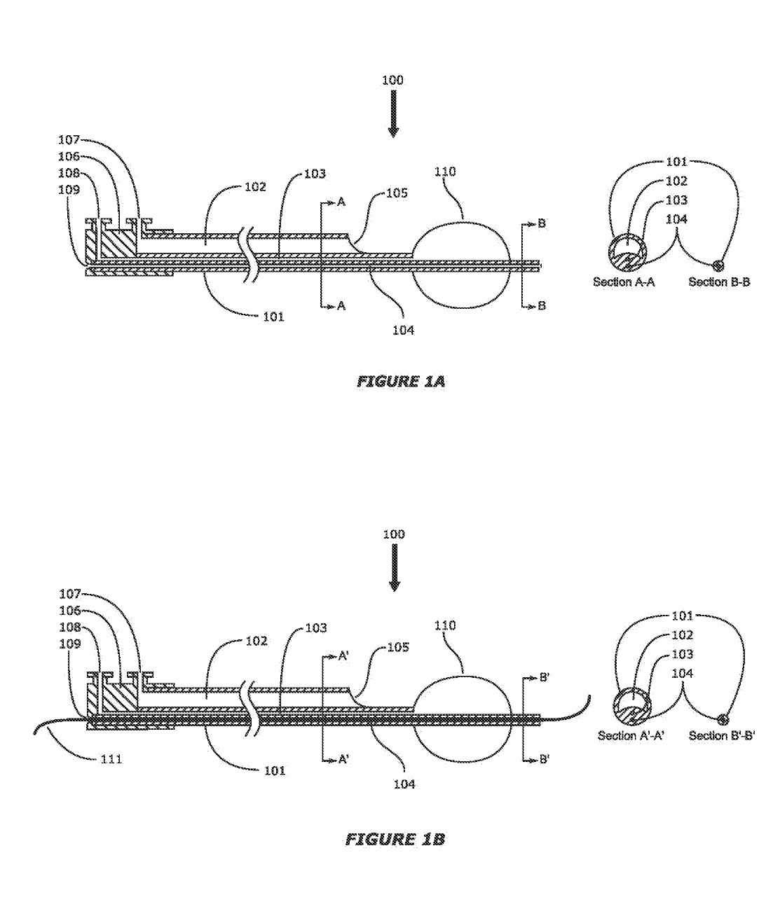

FIGS. 1A-1B depict plan and cross sectional views of a first exemplary embodiment of a balloon catheter 100 that comprises multiple lumens as shown in cross-sections views Section A-A and Section A′-A′. Elongate member 101 has proximal and distal ends comprising a fluid delivery lumen 102, an inflation lumen 103, and a guide wire lumen 104 extending therethrough. Fluid delivery lumen 102 further comprises at least one exit port 105. Exit port 105 may be a single opening at the distal end of fluid delivery lumen 102, as shown in FIGS. 1A and 1B, or it may be a collection of multiple openings (not shown) located at any point along the length and circumference of fluid delivery lumen 102.

The elongate member 101 may be fabricated from materials known in the art including, but not limited to Pebax, nylon, urethane, polyester, polyethylene, polyimide, Teflon, Delrin, PEEK, polycarbonate, polypropylene and combinations thereof. Furthermore, the elongate member 101 may be reinforced with additional materials such as braids, meshes, mandrels, liners, and the like fabricated from materials known in the art including, but not limited to nitinol, stainless steel, titanium, combinations thereof, and the like.

The proximal end of the elongate member 101 may be joined to a manifold 106 using methods known in the art including, but not limited to overmolding, adhesive bonding, ultrasonic welding, crimping, potting, press fitting, and the like. The manifold 106 may be fabricated using molding and/or machining techniques known in the art, and may be fabricated from materials known in the art including, but not limited to polycarbonate, Delrin, nylon, PE, PP, ABS, PEEK, Pebax, PTFE, polymethylmethacrylate, stainless steel, aluminum, titanium, combinations thereof, and the like.

The manifold 106 further includes a fluid injection port 107, inflation port 108, and guide wire insertion port 109. The proximal end of the fluid delivery lumen 102 is in communication with the fluid injection port 107. The proximal end of the inflation lumen 103 is in communication with the inflation port 108, and the proximal end of the guide wire lumen 104 is in communication with the guide wire insertion port 109. The distal end of the inflation lumen 103 is in communication with the interior of expandable member 110.

The expandable member 110, e.g., a balloon, is bonded or otherwise attached to the elongate member 101 at its proximal and distal ends using methods known in the art including, but not limited to, adhesive bonding, crimping, ultrasonic welding, and the like, and may be fabricated from materials known in the art including, but not limited to polyurethane, polyethylene, PET, nylon, combinations thereof, and the like. FIG. 1B depicts plan and cross sectional views of the balloon catheter 100 with a guide wire 111 inserted into and through the guide wire lumen 109. The balloon catheter 100 may also include one or more marker bands or beacons (not shown) that allow for visualization of the device using methods known in the art including, but not limited, to magnetic modalities, ultrasound, electromagnetic imaging, infrared, computed tomography, fluoroscopy, and the like.

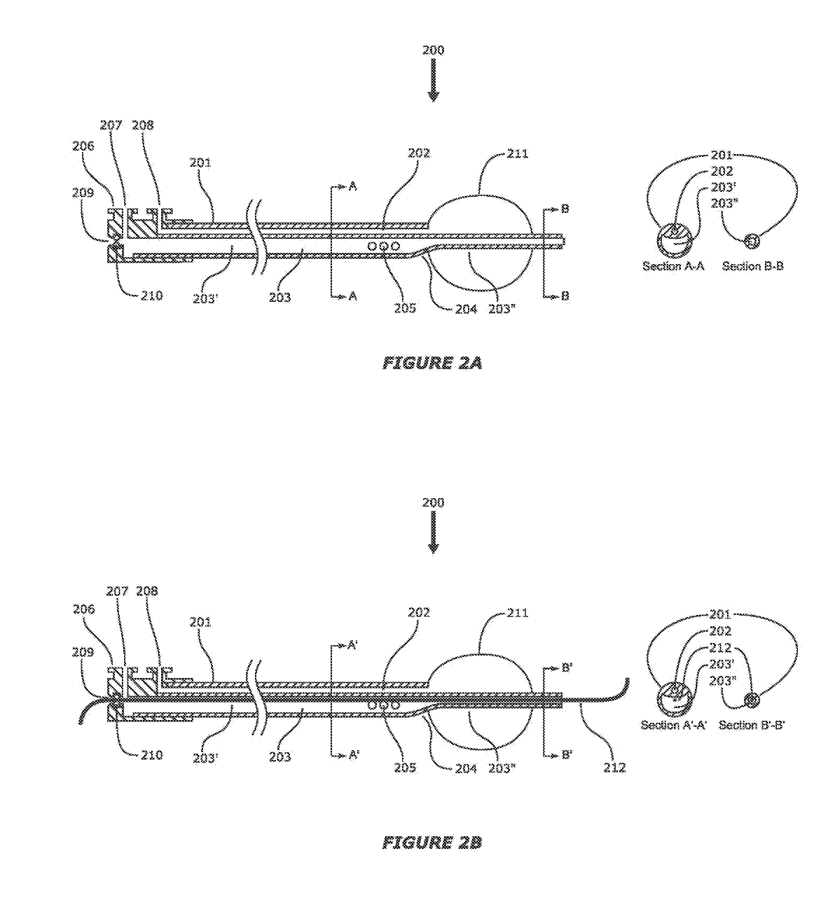

FIGS. 2A-2B depict plan and cross sectional views of a second embodiment of a balloon catheter 200, which may be generally constructed similar to the catheter 100 of FIGS. 1A-1B. Elongate member 201 has proximal and distal ends with an inflation lumen 202 and a fluid delivery/guide wire lumen 203 extending therethrough. The fluid delivery/guide wire lumen 203 further comprises at least one tapered section 204 and at least one exit port 205. In the examples shown in FIGS. 2A and 2B, a single tapered section 204 divides the fluid delivery/guide wire lumen 203 into regions of larger 203′ inner lumen size and smaller 203″ inner lumen diameter. While this configuration is employed to clarify the description of this embodiment, it should be clear to one of skill in the art that multiple tapered sections 204 may be employed to divide contrast delivery/guide wire lumen 203 into multiple regions. Furthermore, it is envisioned that the inner diameter of each of the multiple regions delineated by the multiple tapered sections 204 may have inner diameters or lumens that are of similar or different dimensions and/or different shapes with respect to each other. Likewise, while tapered section 204 is shown as having a proximal region of larger lumen or diameter proximal to a region of smaller lumen or diameter, the opposite configuration may also be provided. The location of tapered section 204 may be at any point between the proximal and distal ends of elongate member 201. Exit port 205 may be a singular opening or a collection of multiple openings located at any point along the length and circumference of the fluid delivery/guide wire lumen 203, or a single port located at the distal tip of the elongate member 201. The catheter 200 may include one or more marker bands or visual indicators (not shown) placed along the length of the elongate member 201 (e.g., a radiopaque marker band positioned inside or within the inflatable infusion element 211 and/or at least one printed marker in the proximal side of the elongate member 201).

The elongate member 201 may be fabricated from materials known in the art including, but not limited to Pebax, nylon, urethane, polyester, polyethylene, polyimide, Teflon, Delrin, PEEK, polycarbonate, polypropylene and combinations thereof. Furthermore, the elongate member 201 may be reinforced with additional materials such as braids, meshes, mandrels, liners, and the like fabricated from materials known in the art including, but not limited to nitinol, stainless steel, titanium, combinations thereof, and the like.

The proximal end of the elongate member 201 may be joined to manifold 206 using methods known in the art including, but not limited to overmolding, adhesive bonding, ultrasonic welding, crimping, potting, press fitting, and the like. The manifold 206 may be fabricated using molding and/or machining techniques known in the art, and may be fabricated from materials known in the art including, but not limited to polycarbonate, Delrin, nylon, ABS, PEEK, Pebax, PTFE, polymethylmethacrylate, stainless steel, aluminum, titanium, combinations thereof, and the like. The manifold 206 further includes a fluid injection port 207, an inflation port 208, a guide wire insertion port 209, and a gasket 210. While the gasket 210 is depicted as an o-ring in FIGS. 2A and 2B, it should be clear to one of skill in the art that any feature for creating a seal at guide wire insertion port 209 may suffice, including but not limited to a Touhy-Borst valve, a septum, a valve fabricated by multiple layers of elastomeric materials, an iris, combination thereof, and the like (not shown). The proximal end of the fluid delivery/guide wire lumen 203 is in communication with the fluid injection port 207 and the guide wire insertion port 209. The proximal end of the inflation lumen 202 is in communication with the inflation port 208. The distal end of the inflation lumen 202 is in communication with the interior of expandable member 211.

Expandable member 211 is bonded to the elongate member 201 at its proximal and distal ends using methods known in the art including, but not limited to adhesive bonding, crimping, ultrasonic welding, and the like, and may be fabricated from materials known in the art including, but not limited to polyurethane, polyethylene, PET, nylon, combinations thereof, and the like. FIG. 2B depicts plan and cross sectional views of the balloon catheter 200 with a guide wire 212 inserted into and through the fluid delivery/guide wire lumen 203. As can be seen in FIG. 2B, the gasket 210 is sized to seal against the guide wire 212 and prevent fluid injected through the fluid delivery port 207 and into the fluid delivery/guide wire lumen 203 from flowing through the guide wire port 209. The region of the fluid delivery/guide wire lumen 203 of smaller inner diameter 203″ is sized to closely approximate the guide wire 212.

Thus, the insertion of the guide wire 212 into region 203″ may significantly reduce the cross sectional area of the fluid delivery/guide wire lumen 203 available to flow as compared to the size of the exit port 205, thus directing the flow of fluid preferentially out of the exit port 205. While the outer diameter of the guide wire 212 and the smaller inner diameter 203″ of the fluid delivery/guide wire lumen 203 are shown to be in close approximation in FIG. 2B, it will be appreciable to one of skill in the art that the degree of flow of fluid out of the exit port 205 may be controlled by varying the size and/or number of exit ports 205, the outer diameter of the guide wire 212, and the smaller inner diameter 203″ of the fluid delivery/guide wire lumen 203. The balloon catheter 200 may further include one or more marker bands or beacons (not shown) that allow for visualization of the device using methods known in the art including, but not limited to magnetic modalities, ultrasound, electromagnetic imaging, infrared, computed tomography, fluoroscopy, and the like.

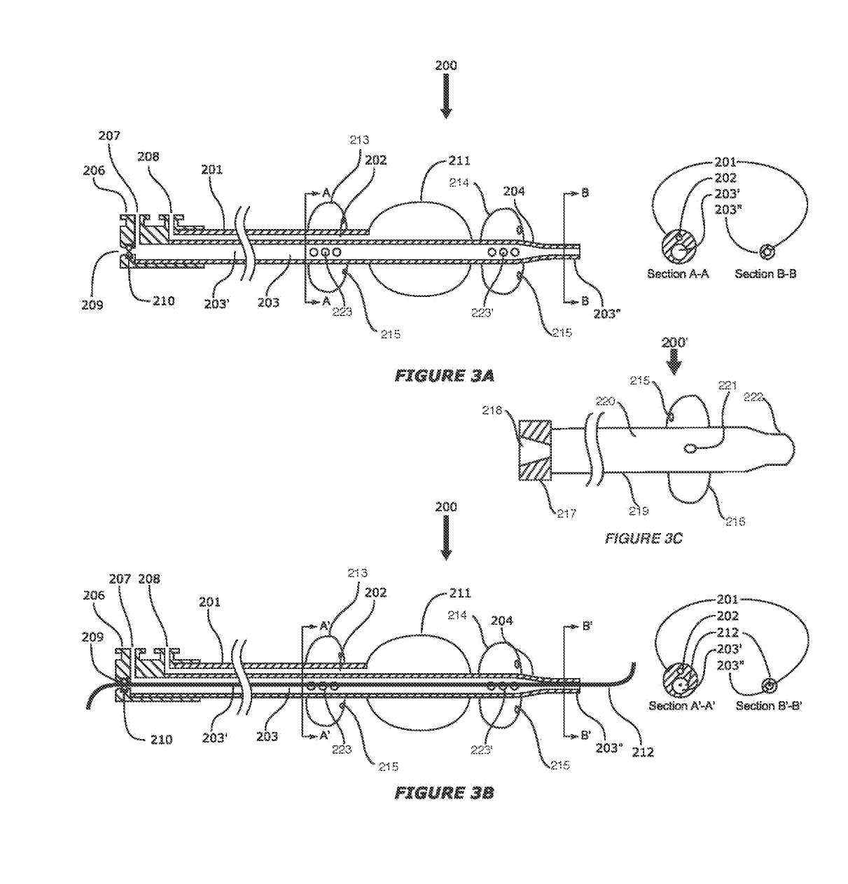

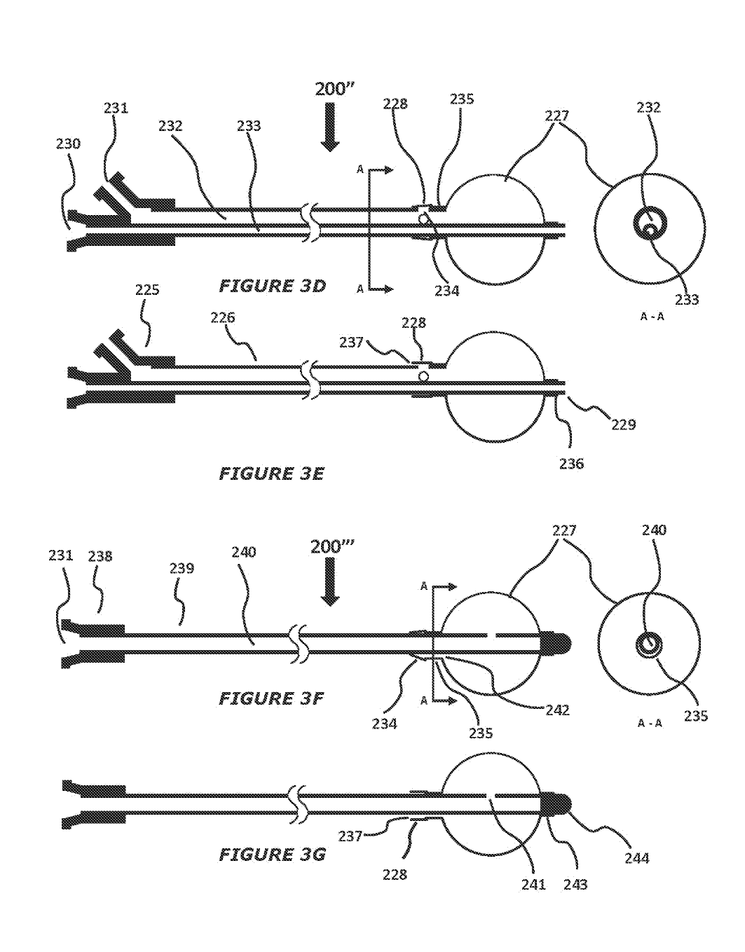

FIGS. 3A and 3B depict an alternative embodiment of balloon catheter 200 wherein the tapered section 204 is located distal to the distal end of the expandable member 211 and a cover or shield, for example an inflatable balloon or other infusion element 213, 214, is provided over a respective set of transit ports or openings 223, 223′ with each cover or shield including one or more exit ports 215. In the embodiment shown, at least one transit port 223′ is located distal of the expandable member 211 and covered or shielded by a first inflatable infusion element 214 and at least one transit port 223 is located proximal of the expandable member 211 covered or shielded by a second inflatable infusion element 213. Both inflatable inflation elements 213 and 214 include at least one infusion exit port or opening 215 that aims and projects infused fluid along the direction of the blood vessel or lumen. The catheter 200 may include at least one marker band or visual indicator (not shown) placed along the length of the elongate member 201 (e.g., a radiopaque marker band positioned inside or within the inflatable infusion elements 213 and 214 and/or at least one printed marker located along the proximal side of the elongate member 201, not shown).

The inflatable infusion elements 213 and 214 may be fabricated from compliant and/or non-compliant, reinforced and/or unreinforced plastics, elastomers and/or composites thereof, including but not limited to: polyurethane, PEBAX, PET, nylon, PE, silicone rubber, C-flex and the like. The inflatable infusion elements 213 and 214 may be fabricated using processes including but not limited to blow molding, dipping, spraying, injection molding, extruding and the like. The transit ports 223, 223′ and the directional infusion exit ports 215 may be formed using processes including but not limited to drilling, laser cutting, die punching, skiving, and the like. The first inflatable infusion element 214 and the second inflatable infusion element 213 may each include at least one directional infusion exit port 215 oriented to aim the fluid jet, e.g., distally simultaneous with the radial expansion of the inflatable infusion elements 214 and 213.

Alternatively, the exit port(s) 215 may be oriented or otherwise configured such that the fluid jet is ejected from the exit port(s) 215 may be aimed proximally or distally or any combination thereof, e.g., by positioning the directional infusion exit port 215 on the appropriate sides of the inflatable infusion elements 213 and 214. The directional infusion port 215 may also be complimented or replaced by providing an opening located at the interface between the inflatable infusion elements 213 and/or 214 and the outer surface of the elongate member 201. The inflatable infusion elements 214 and 213 may expand to a diameter configured to engage a surrounding wall of a vessel or lumen, e.g., to cause occlusion of the vessel or lumen.

The number of directional infusion exit ports 215 and the dimensions of the directional infusion exit ports 215 may be sized to allow pressure buildup within the inflatable infusion elements 214 and 213 while substantially simultaneously facilitating optimal infusion flow rate and adequate vessel occlusion. Ideally, the pressure built up within the inflatable infusion elements 214 and 213 would be low enough to prevent damage or trauma to surrounding vessel walls or lumens. The flow rate and direction of fluid through the directional infusion exit ports 215 may be controlled by varying the size, the number, the longitudinal and radial location, and/or the geometry of the directional infusion exit ports 215, the inner diameter of the region of smaller inner diameter 203″ of the fluid delivery/guide wire lumen 203, and/or the outer diameter of guide wire 212. Alternatively, the expanded diameter of the inflatable infusion elements 213 and 214 may be reduced to obviate vessel or lumen occlusion to thereby maintain blood perfusion.

Alternatively the catheter 200 may include only one inflatable infusion element (not shown). For example, the catheter 200 may include only inflatable infusion element 213 and expandable member 211; or may comprise only inflatable infusion element 214 and expandable member 211. In yet another alternative, the catheter 200 may include no inflatable infusion elements 214 and/or 213 such that infused fluids may exit directly to the bloodstream or vessel or lumen via the transit ports 223′ and/or 223.

In these various embodiments shown in FIGS. 3A and 3B, the insertion of a guide wire 212 into the fluid delivery/guide wire lumen 203 creates a restriction to flow at the region of smaller inner diameter 203″ of fluid delivery/guide wire lumen 203. The restriction in turn creates paths of lesser resistance for fluids introduced into the fluid delivery/guide wire lumen 203 through the proximal transit port 223 and the distal transit port 223′ and subsequently into the inflatable infusion elements 214 and 213 such that the infusion fluid may exit to the bloodstream or vessel or lumen via the directional infusion exit ports 215.

FIG. 3C shows a side view of an alternative embodiment of a balloon catheter 200′ generally constructed similar to the catheter 200 of FIG. 3A but including only a single inflatable infusion element 216 having a directional infusion exit port 215 positioned on the proximal side of the inflatable infusion element 216. Similar to other embodiments, the catheter 200′ may include at least one marker band and/or visual indicator placed along the length of the catheter 200′ (e.g., a radiopaque marker band positioned inside or within the inflatable infusion element 216 or printed marker located along the proximal side of the shaft, not shown).

The catheter 200′ lacks an expandable treatment member 211 such as that shown in FIG. 3A and may be used for diagnostic imaging of a stenosis that is proximal to the inflatable infusion element 216. For example, in dialysis patients, the catheter 200′ may allow visualization of an arterial anastomosis via retrograde opacification using contrast without exposing the operator to X-rays or radiation. The catheter 200′ may include a proximal hub 217 with an infusion entry port 218 that communicates with an elongate shaft member 219 having a lumen 220 and terminating at distal tip 222. The shaft member 219 includes at least one transit port 221 in a side wall thereof that communicates between the lumen 220 and the interior of the inflatable infusion element 216 such that infused fluid may exit to the bloodstream or vessel or lumen via the directional infusion exit port(s) 215. The infusion flow rate and pressure build up within the inflatable infusion element 216 may be controlled using the constructions and/or methods described previously.

An alternative embodiment to that shown in FIG. 3C may include a through lumen to accommodate a guide wire such that the distal tip 222 is open and includes a guide wire exit port (not shown). As described previously, the open ended distal tip 222 may be sized to create a path of least resistance towards the transit port 221. The materials and processes used to fabricate this embodiment may be generally similar to those described elsewhere herein with reference to FIGS. 3A and 3B.

In an exemplary embodiment, a method for using the catheter 200′ in a vascular setting may include: (1) placing a distal tip of an intravascular procedure sheath into a body lumen (not shown), e.g., by standard puncture and placement, (2) inserting the catheter 200′ through a hemostasis valve of the placed intravascular sheath and advancing the catheter 200′ until the inflatable infusion element 216 exits the distal tip of the intravascular sheath via confirmation, e.g., visually using X-ray confirmation of a radiopaque marker (not shown) relative to the distal tip of the intravascular sheath and/or confirmation of a positioning marker relative to the valve of the intravascular sheath, (3) attaching a contrast filled syringe or other injection device to the proximal hub 217, and (4) delivering contrast or contrast/saline solution through the infusion entry port 218 with sufficient injection force so as to radially expand the inflatable infusion element 216, occluding blood flow within the vessel while simultaneously providing retrograde opacification of the vessel via fluid infusion through the proximal facing directional infusion exit port 215.

Alternative embodiments to catheter 200′ are shown in FIGS. 3D-3G, which include cross-sectional views of catheters 200″ and 200′″. Each of these catheters 200″ 200′″ include at least one directional infusion exit opening 237 that directs infusion of fluid proximally and an inflatable infusion element 227 that may partially or totally occlude a vessel or artery during fluid infusion or injection. The inflatable infusion element 227 may be a balloon fabricated from compliant and/or non-compliant, reinforced and/or unreinforced plastics, elastomers and/or composites thereof, including but not limited to: polyurethane, PEBAX, PET, nylon, PE, silicone rubber, C-flex and the like. The inflatable infusion element 227 may be fabricated using processes including but not limited to blow molding, dipping, spraying, injection molding, extruding and the like or combinations thereof.

Referring to FIGS. 3D and 3E, the catheter 200″ may include a multi-lumen elongate member 226 having a proximal end, a distal end, a guide wire lumen 233 extending there between, and an inflation lumen 232 extending from the proximal end at least partially to the distal end, which also serves as the fluid delivery lumen. A manifold 225 is attached to the proximal end of the elongate member 226 that includes a guide wire port 230, and an inflation port 231, which also serves as a fluid delivery port. The inflation port 231 directly communicates with the inflation lumen 232 and the guide wire port 230 directly communicates with the guide wire lumen 233.

An inflatable infusion element 227 is attached to a distal portion of the elongate member 226 such that a wall of the elongate member 226 defining the distal end of the inflation lumen 232 terminates and is attached to a proximal leg 235 of the inflatable infusion element 227. Thus, the inflation lumen 233 is in direct communication with the interior of the inflatable infusion element 227. The guide wire lumen 233 extends past the distal leg 236 of the inflatable infusion element 227 such that a distal leg 236 of the inflatable infusion element 227 is attached to the elongate member 226 adjacent a tip thereof 229 including an outlet port for the guide wire lumen 233. The elongate member 226 includes at least one transit port 234 disposed at a distal section of the inflation lumen 233 proximal to the proximal leg 235 of the inflatable infusion element 227. The transit port 234 may be formed using processes including but not limited to drilling, laser cutting, die punching, skiving, and the like.

A compliant tubular member 228 is provided as a cover or shield positioned over the transit port(s) 234, e.g., to act as a one-way valve allowing fluid to flow out of the transit port(s) 234 and preventing fluid to flow into the transit port(s) 234. The compliant tubular member 228 may be made of flexible plastic or elastomeric materials such as latex, silicone rubber, polyurethanes, CFLEX, and the like or combinations thereof. A distal end of the compliant tubular member 228 is positioned and circumferentially joined to the distal end of the elongate member 226 distal to transit port 234 and/or to the proximal leg 235 of the inflation infusion element 227. A proximal end of the compliant tubular member 228 terminates at a location proximal of transit port 234 (i.e., between the transit port 234 and the proximal end of the elongate member 226). The proximal end of the compliant tubular member 228 may be partially attached over the elongate member 226 (e.g., at one or more locations around an outer wall or surface of the elongate member 226) or alternatively, may not be attached at all but simply surround the outer wall of the elongate member 226.

In this manner, the proximal end of the compliant tubular member 228 may provide a mechanical seal over the transit port 234, which may selectively open to create a directional infusion exit opening 237. The injection or delivery of fluid into the inflation lumen 232 (from the inflation port 231) generates pressure within the inflation fluid path, thereby inflating the inflatable infusion element 227. Further injection of fluid causes the internal pressure to increase and at a predetermined elevated pressure threshold, the compliant tubular member 228 expands or stretches as depicted in FIG. 3E, e.g., such the proximal end of the compliant tubular member 228 separates at least partially from the outer wall of the elongate member 226, allowing the fluid to pass through the transit port 234 and out of the directional infusion exit opening 237. As soon as the fluid injection or delivery is completed, i.e., the pressure is reduced below the predetermined threshold, the compliant tubular member 228 contracts or recoils back to mechanically re-seal the transit port 234.

When a negative pressure is applied to the inflation lumen 232 of the catheter 200″ (e.g., using a syringe attached to inflation port 231 to create an internal vacuum), the proximal end of the compliant tubular member 228 may be drawn against the outer wall of the elongate member 226 to provide a substantially fluid tight mechanical seal, which may prevent external fluid such as air or blood from being suctioned into the inflation lumen 232 from the region surrounding the catheter 200″, and also allows the inflatable infusion element 227 to be deflated into a collapsed state, reducing its profile to ease insertion and withdrawal of the catheter 200″ through an introducer or other sheaths, such as those typically used for vessel access.

Alternatively, the proximal leg 235 of the inflatable infusion element 227 may act as a replacement for compliant tubular member 228, e.g., by extending the length of the proximal leg 235 proximally beyond the transit port 234 (not shown). The extended length of the proximal leg 235 may be partially attached to the outer wall of the elongate member 226, e.g., at one or more points around the circumference of the elongate member 226 while providing one or more circumferential gaps between the extended proximal leg 235 and the outer wall. Alternatively, the extended proximal leg 235 may not be attached at all to the elongate member 226, e.g., over or proximal to the transit port 234, such that the extended proximal leg 235 may expand to create a directional infusion exit opening when exposed to pressure within the infusion lumen 233 beyond a predetermined threshold. In yet another embodiment, the compliant tubular member 228 may be omitted from the catheter 200″ of FIGS. 3D-3E, leaving the transit port 234 exposed to the space or environment external to the catheter 200″.

FIGS. 3F-3G depict a cross sectional view of catheter 200′″ illustrating another alternative embodiment that includes an infusion exit opening that directs fluid in the proximal direction. The catheter 200′″ includes a single lumen elongate member 239 having a lumen 240 that serves two purposes, the first being for inflating inflatable infusion element 227 and the second being for delivering fluid such as contrast agents or medications into a vessel or lumen within which the catheter 200′″ is introduced. The proximal leg 235 is partially attached over the elongate member 239 as shown in FIG. 3F and cross section A-A of FIG. 3F, leaving a channel 242 between the outer wall of the elongate member 239 and inner wall of the proximal leg 235.

The catheter 200″ is generally similar in function and construction to the catheter 200′ shown in FIG. 3C except that the location of where the fluid is exiting from is relocated and replaced by a directional infusion exit opening 237 that is in communication with channel 242. This is in contrast to the directional infusion exit port 215 shown in FIG. 3C, which is located on the inflatable segment of the inflatable infusion element 216.

Returning to FIG. 3F, a compliant tubular member 228 is provided adjacent to the proximal leg 235, which functions similarly to the compliant tubular member described with reference to the catheter 200″ shown in FIGS. 3D-3E. The distal end of the compliant tubular member 228 may be circumferentially joined to the proximal leg 235. Alternatively, the end of the proximal leg 235 may act as a replacement of compliant tubular member 228, e.g., by extending its length towards and over the proximal side of the transit port 234 and having the extended length partially attached to the elongate member 226 or not attached at all, as described previously. In yet another embodiment, the compliant tubular member 228 may be removed, leaving the channel 242 exposed to the space or environment external to the catheter 200′″.

Another alternative is to use a valve mechanism (not shown) or other form of shielding or covering associated with the transit port 234, in place of the compliant tubular member 228. This valve mechanism may provide one-way directionality of fluid flow and/or may open at a predefined infusion pressure, thus having similar function as the compliant tubular member 228. In still another alternative embodiment, the location where the fluid exits from the catheter 200′″ may be located distal of the inflatable infusion element 227 expandable member, e.g., to direct the fluid infusion in a distal direction, i.e., distally beyond the distal tip of the elongate member 239 (not shown).

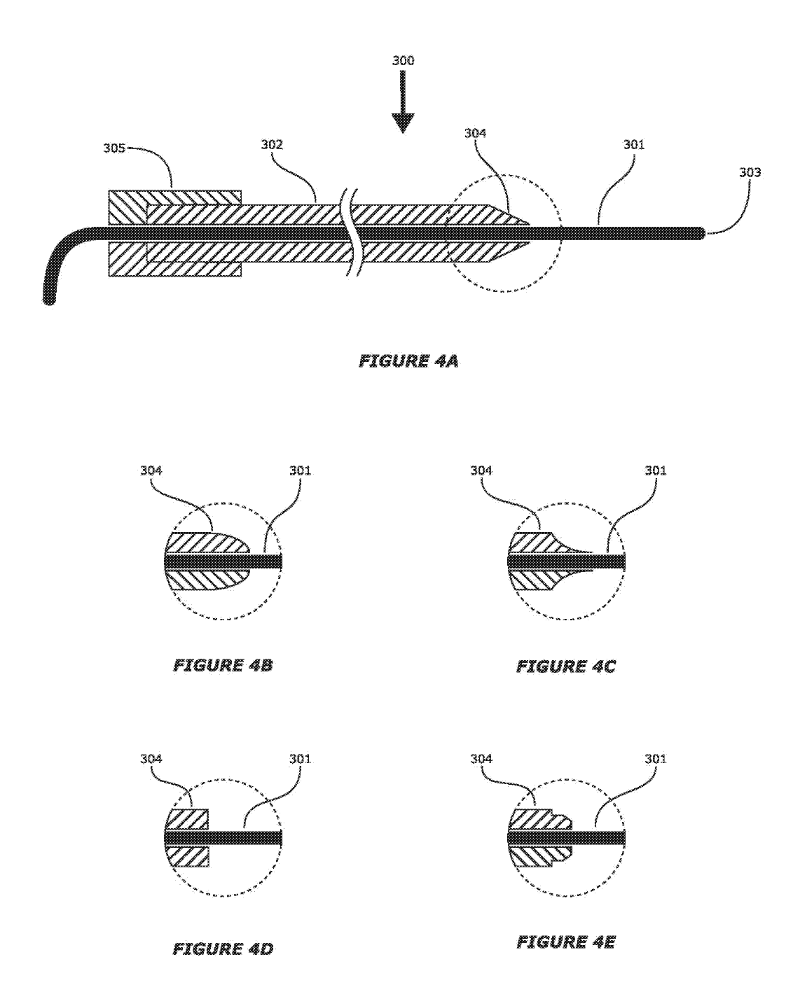

Turning to FIGS. 4A-4E, an exemplary embodiment of a reinforced guide wire 300 is shown that comprises a guide wire 301 with proximal and distal ends and a removable jacket 302. The guide wire 301 may be fabricated from materials known in the art including, but not limited to, stainless steel, platinum, titanium, nitinol, combination thereof, and the like. The guide wire 301 may optionally be configured as an inner core wire and outer coil wrap wherein the inner core wire may or may not be connected to the distal end of the outer coil wrap (not shown). Alternatively, the guide wire 301 may include a nitinol core coated with a polymer such as polyurethane or PTFE (also not shown). The distal tip 303 of the guide wire 301 may be shaped in an atraumatic geometry, such as a hemisphere, dome, or the like. The surface of the guide wire 301 may be coated (not shown) with hydrophilic or hydrophobic and/or antithrombogenic materials, plated with gold or platinum, and/or other otherwise modified to obtain properties that differ from those of the underlying material.

The removable jacket 302 comprises proximal and distal ends and at least one lumen therethrough, and is arranged coaxially about the guide wire 301. The distal region 304 of the removable jacket 302 may be tapered as shown in FIG. 4A. Furthermore, the removable jacket 302 may be radially symmetric or radially asymmetric. The removable jacket 302 may be formed from materials known in the art including, but not limited, to stainless steel, platinum, nitinol, Pebax, nylon, Delrin, polymethymethacrylate, polyurethane, polyimide, PTFE, combinations thereof, and the like. The shape of the distal region 304 may be convex (e.g., as shown in FIG. 4B), concave (e.g., as shown in FIG. 4C), stepped or blunt (e.g., as shown in FIG. 4D), or some combination thereof (e.g., as shown in FIG. 4E). It should be clear to one of skill in the art that other alternative shapes for the distal region 304 may be considered and fabricated.

The removable jacket 302 may have a different or similar stiffness than guide wire 301. Moreover, the removable jacket 302 may comprise of one stiffness along its length or may have sections of different stiffnesses along its length. The proximal end of the removable jacket 302 may be joined to a handle 305, e.g., using one or more features known in the art including, but not limited to, adhesive bonding, soldering, welding, ultrasonic welding, press fitting, threading/tapping, snap fitting, combinations thereof, and the like. The handle 305 may be fabricated using methods known in the art including machining, molding, forging, and the like out of materials known in the art including, but not limited to, stainless steel, titanium, polycarbonate, polymethylmethacrylate, Pebax, ABS, delrin, nylon, polyurethane, and the like. The handle 305 and/or removable jacket 302 may also include a sculpted, grooved, scalloped, turned, or otherwise featured outer surface, e.g., to ease holding and/or handling. The handle 305 and/or removable jacket 302 may also include a pad or pads fabricated from materials known in the art including, but not limited to silicone rubber, polyurethane, polyethylene, flexible polyvinylchloride, combinations thereof, and the like, e.g., to aid in holding and handling. The handle 305 may be immobile relative to the removable jacket 302 or may be free to move longitudinally and/or radially with respect to the removable jacket 302.

The removable jacket 302 and guide wire 301 may be separable and reversibly joined to each other, and/or may be able to move longitudinally and/or radially relative to each other. For example, one or more features (not shown) may be provided to reversibly lock the removable jacket 302 to the guide wire 301 (not shown in FIG. 4A), e.g., on the handle 305, removable jacket 302, guide wire 301, or a combination thereof. The feature(s) to reversibly lock the removable jacket 302 to the guide wire 301 may comprise mechanisms known in the art including, but not limited to, a Touhy-Borst valve, a living hinge, detents, a ball and spring mechanism, a key and track mechanism, a tap and screw, clamp, combinations thereof, and the like (not shown).

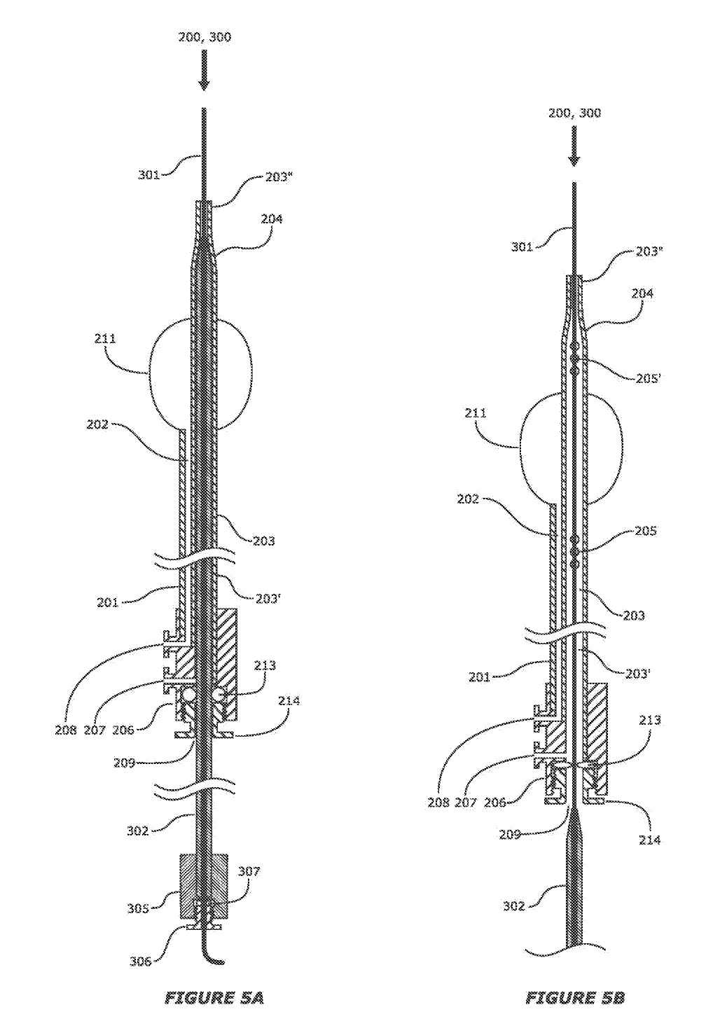

FIGS. 5A and 5B depict an exemplary embodiment of a balloon catheter 200 and an exemplary embodiment of a reinforced guide wire 300. The removable jacket 302 of the reinforced guide wire 300 may be either resident within the fluid delivery/guide wire lumen 203 of the balloon catheter 200 (FIG. 5A) or retracted proximally out of the proximal end of the fluid delivery/guide wire lumen 203 of the balloon catheter 200 (FIG. 5B).

Manifold 206 on the proximal end of the balloon catheter 200 has been modified to further comprise a larger gasket 213 and threaded member 214. Furthermore, the inner diameter of the guide wire insertion port 209 has been increased in size to accommodate the outer diameter of the removable jacket 302 of the reinforced guide wire 300. The handle 305 of the reinforced guide wire 300 has been modified to further comprise a gasket 307 and threaded member 306.

In FIG. 5A, the guide wire 301 and removable jacket 302 are coupled to each other via a threaded member 306 compressing a gasket 307 against the handle 305 and guide wire 301. The reinforced guide wire 300 is coaxially arranged within the fluid delivery/guide wire lumen of the balloon catheter 200, with the distal tip of the removable jacket 302 butting against the tapered section 204 of the balloon catheter 200. Threaded member 214 is configured such that the gasket 213 provides a seal between the manifold 206 and the outer surface of the removable jacket 302. This arrangement provides the guide wire 301 and the balloon catheter 300 with the additional stiffness of the removable jacket 302 and may allow for easier advancement of the balloon catheter 300 into position near a targeted arterial lesion.

In FIG. 5B, the guide wire 301 has been decoupled from the removable jacket 302, and the removable jacket 302 has been retracted proximally out of the proximal end of the fluid delivery/guide wire lumen 203 of the balloon catheter 200. The threaded member 214 has been tightened such that the gasket 213 provides a seal between the manifold 206 and the outer surface of the guide wire 301. This arrangement enables the injection of a solution of contrast into fluid injection port 207, through the fluid delivery/guide wire lumen 203, and out of the exit ports 223 and 223′. Alternatively (not shown), the removable jacket 302 and guide wire 301 may be decoupled from each other while in the configuration depicted in FIG. 5A, and the guide wire 301 may be advanced distally. In this scenario, the gasket 213 may maintain a seal between the manifold 206 and the outer surface of the removable jacket 302.

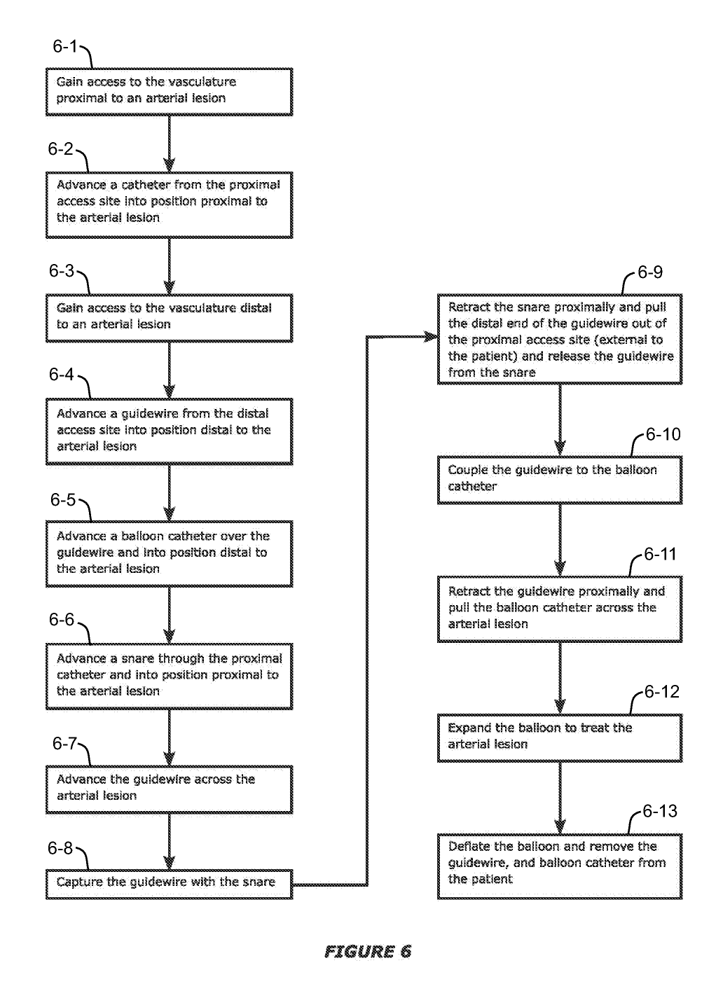

FIG. 6 is a flowchart illustrating a general method for treating arterial lesions (e.g., lesions below the knee treated using a percutaneous trans-pedal access point distal to an obstruction or lesion) using embodiments described herein. At Step 6-1, access to the patient's vasculature is obtained at a point proximal to the arterial lesion or obstruction (usually at the common femoral artery or superficial femoral artery) and, at Step 6-2, a catheter (e.g., a guide catheter or sheath) is advanced distally from the proximal access point to a position proximal to the arterial lesion or obstruction. At Step 6-3, access to the patient's vasculature is then obtained at a point distal to the arterial lesion or obstruction using a guide catheter or sheath and, at Step 6-4, a guide wire is advanced into a position distal to the arterial lesion from this distal access site. At Step 6-5, a balloon catheter is then advanced over the guide wire and into a position distal to the arterial lesion.

At Step 6-6, a snare (or other similar device that is capable of capturing a guide wire) is inserted and directed through the proximal catheter (e.g., guide catheter or sheath) and into a position proximal to the arterial lesion. At Step 6-7, the guide wire is then placed across the arterial lesion into a position wherein the distal end of the guide wire is advanced and positioned proximal to the proximal edge of the arterial lesion. At Step 6-8, the snare is then advanced distally towards the arterial lesion and used to capture the distal end of the guide wire. At Step 6-9, the snare is then retracted proximally to pull the guide wire extracorporeally. The guide wire portion external to the patient is then released from the snare.

At Step 6-10, the balloon catheter and guide wire are then coupled to each other using methods that include, but are not limited to, those described herein (e.g., through the use of a locking guide wire, and/or a balloon catheter with locking feature(s), or the combination of a catheter with a tapered lumen and a reinforced guide wire with a removable jacket, etc.). At Step 6-11, the guide wire is then retracted such that a tensile load placed on the guide wire will be transmitted to the balloon catheter. Further retraction of the guide wire pulls the balloon catheter across or within the arterial lesion. This step may be performed under fluoroscopic guidance, e.g., making reference to marker band(s) on the balloon catheter relative to the surrounding anatomy to ensure desired positioning within or across the lesion. At Step 6-12, the balloon is then expanded to treat the treat lesion. After treatment, at Step 6-13, the balloon is then deflated and the guide wire and balloon catheter are removed from the patient en bloc, serially, or any combination thereof. It should be clear to one of skill in the art that changes in the order of operations of these exemplary steps (e.g., positioning the proximal catheter and/or the snare prior to obtaining access to the patient's vasculature at a point distal to the arterial lesion, etc.) are also contemplated.

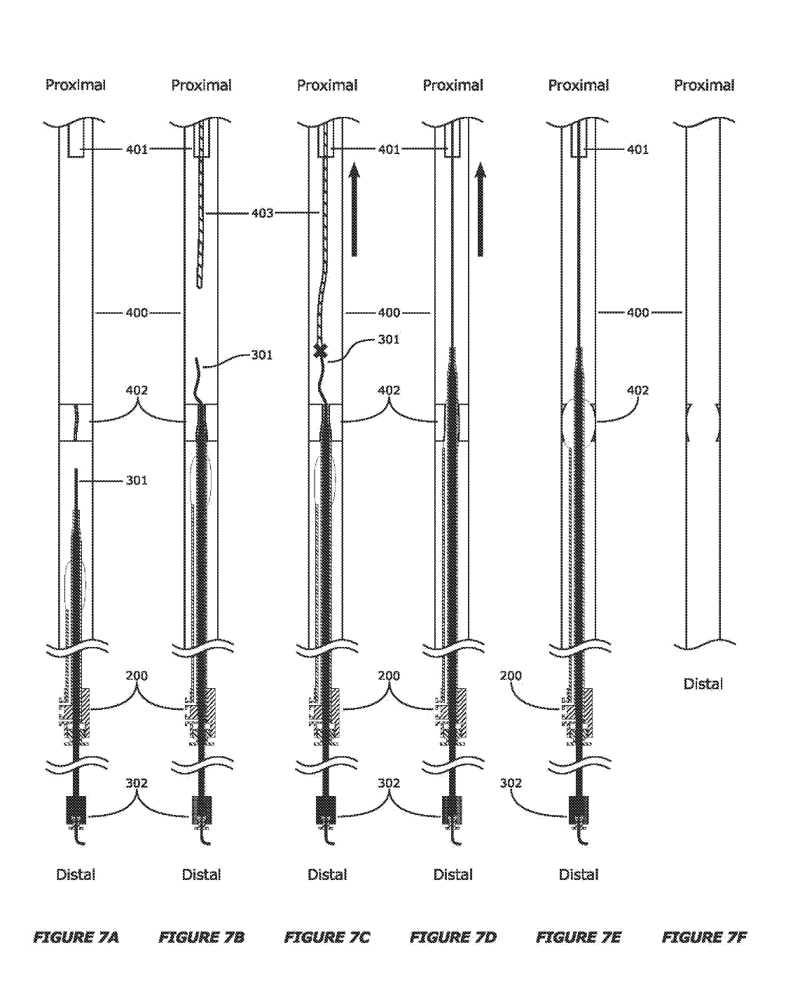

FIGS. 7A-7F depict schematic illustrations of an exemplary method for treating arterial lesions. The images in FIGS. 7A-7F are intended to illustrate the steps in treating an arterial lesion and the various lengths, distances, diameters, depicted in the images are not representative of clinical anatomy. The extension of the general method described in FIGS. 7A-7F to the clinical treatment of specific arterial lesions should be clear to one of skill in the art.

FIG. 7A shows a lesion or plaque 402 substantially blocking an artery 400. The lesion 402 is approached from the distal vasculature with the balloon catheter 200 and reinforced guide wire 300 (e.g., such as that shown in FIG. 4A) including a guide wire 301 and removable jacket 302. A guide catheter or sheath 401 is introduced and positioned proximal to the arterial lesion 402. The guide wire 301 is decoupled from the removable jacket 302 in preparation for crossing the arterial lesion 402 from the distal side of the lesion 402. FIG. 7B shows the guide wire 301, supported by the balloon catheter 200 and the removable jacket 302, advanced across the arterial lesion 402.

As shown in FIG. 7B, a snare 403 is introduced and advanced along the guide catheter or sheath 401 to capture the distal end of the guide wire 301. FIG. 7C shows the snare 403 that has captured the distal end of the guide wire 301 (as denoted by an “X” in the drawing). In this configuration, a tensile load applied to the snare 403 pulls the guide wire 301 in the proximal direction as shown by the arrow in FIG. 7C and withdrawn extracorporeally through the lumen of the guide catheter or sheath 401. At this point in the procedure, the guide wire is coupled to the removable jacket 302, joining the two components into a single unit. The distal end of the guide wire 301 external to the patient is pulled, applying a tensile load transmitted along the length of the guide wire 301. This load is communicated to the removable jacket 302, and in turn through the removable jacket 302 to the balloon catheter 200 through the interaction between the taper of the removable jacket 302 and the taper in the fluid delivery/guide wire lumen of the balloon catheter 200 (shown in detail in FIG. 5A), pulling the expandable member of the balloon catheter 200 within or across the arterial lesion 402, as shown in FIG. 7D. The arrow in FIG. 7D shows the direction of the pull.

The expandable member of the balloon catheter 200 is then expanded against the arterial lesion, as shown in FIG. 7E. The balloon catheter 200 and reinforced guide wire 300 (FIG. 4A) may be removed en bloc, in sequence, or any combination thereof, resulting in the treated arterial lesion 402 shown in FIG. 7F. It should be clear to one of skill in the art that the removable jacket may be retracted out of the fluid delivery/guide wire lumen (as shown in FIG. 5B) at any point during the procedure to allow fluid delivery through the exit ports of the fluid delivery/guide wire lumen of balloon catheter 200 and the recording of a angiographic image of the vasculature surrounding the arterial lesion 402. Furthermore, while the exemplary method shown in FIGS. 7A-7F illustrates the treatment of an arterial lesion 402 wherein the balloon catheter 200 and reinforced guide wire 300 approach the lesion 402 from the distal vasculature, the reverse approach (e.g., with the balloon catheter 200 and reinforced guide wire 300 approaching the lesion 402 from the proximal vasculature and the snare 403 approaching from the distal vasculature) is also contemplated.

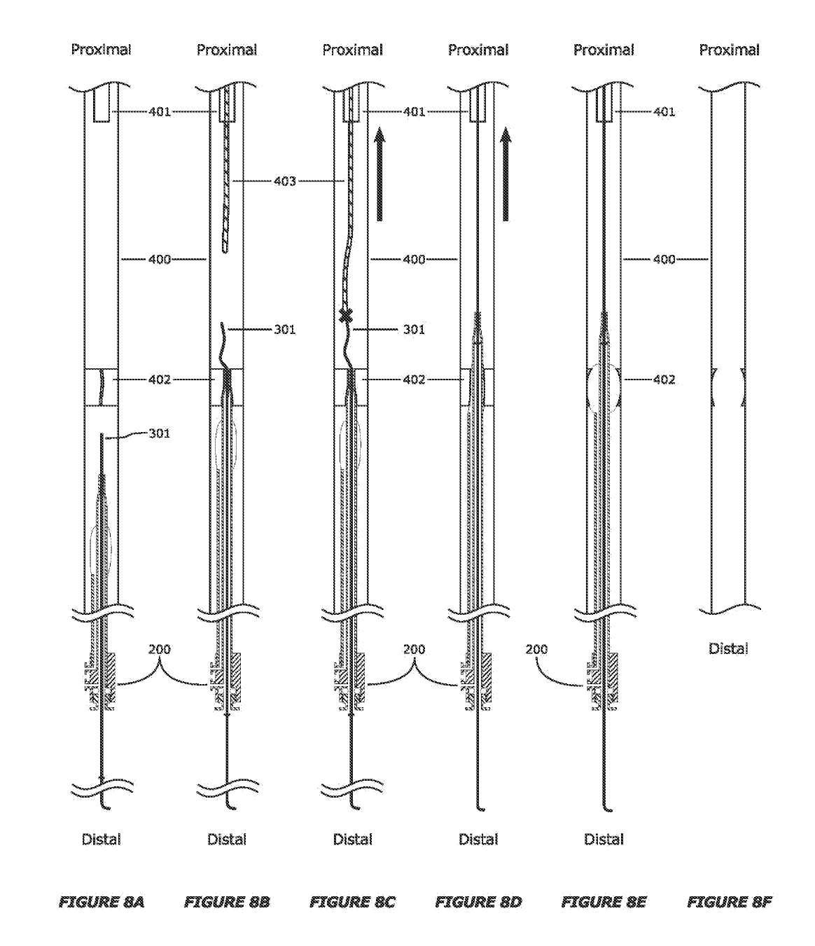

FIGS. 8A-8F depict schematic illustrations of an alternative method for treating arterial lesions using a balloon catheter 200 (which may be any of the embodiments described herein) and a guide wire 301 with a segment of increased diameter located at a proximal location along the guide wire 301. The images in FIGS. 8A-8F are intended to illustrate the steps in treating an arterial lesion 402 and the various lengths, distances, diameters, depicted in the images are not representative of clinical anatomy. The extension of the general method described in FIGS. 8A-8F to the clinical treatment of specific arterial lesions should be clear to one of skill in the art.

FIG. 8A shows a lesion 402 or plaque substantially blocking an artery. The lesion 402 is approached from the distal vasculature with the balloon catheter 200, and a guide catheter or sheath is positioned proximal to the arterial lesion 402. FIG. 8B shows the guide wire 301, supported by the balloon catheter 200, advanced across the arterial lesion 402 and a snare 403 positioned to capture the distal end of the guide wire 301. FIG. 8C shows the snare 403 capturing the guide wire 301 of the reinforced guide wire (as denoted by an “X” in the drawing).

In this configuration, a tensile load applied to the snare 403 will be communicated to the guide wire 301, pulling the guide wire 301 in the proximal direction as shown by the arrow in FIG. 8C and ultimately position distal end of the guide wire 301 extracorporeally. With the distal end of the guide wire 301 external to the patient, the physician applies a tensile load to the distal end of the guide wire 301, pulling the guide wire 301 along the lumen of the balloon catheter 200 until the guide wire 301 segment of larger diameter contacts the taper transition in the fluid delivery/guide wire lumen of the balloon catheter. Further application of tensile force on the distal end of the guide wire 301 pulls the expandable member of the balloon catheter 200 across the arterial lesion 401, as shown in FIG. 8D. The direction of the force is shown by the arrow in FIG. 8D.

The expandable member is then expanded one or more times against the arterial lesion 402, as shown in FIG. 8E. The balloon catheter 200 and guide wire 301 may be removed en bloc, in sequence, or any combination thereof, resulting in the treated arterial lesion 402 shown in FIG. 8F. Furthermore, while the exemplary method shown in FIGS. 8A-8F illustrates the treatment of an arterial lesion 402 wherein the balloon catheter 200 and guide wire 301 approach the lesion 402 from the distal vasculature, the reverse approach (e.g. the balloon catheter 200 and guide wire 301 approaching the lesion 402 from the proximal vasculature and the snare 403 approaching from the distal vasculature) is also contemplated.

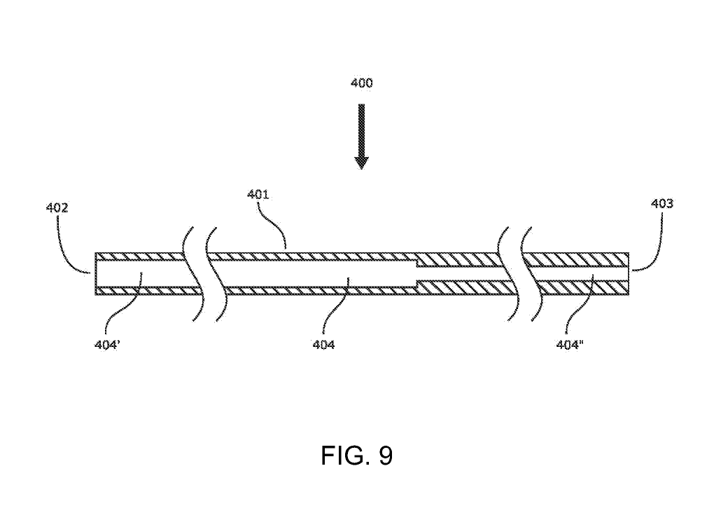

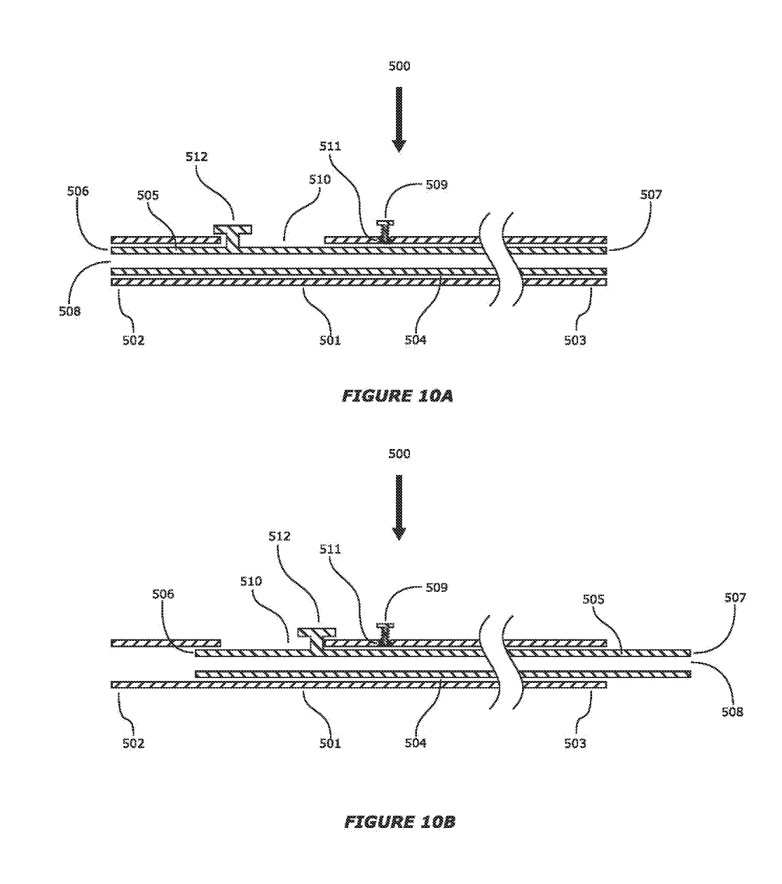

FIG. 9 depicts an exemplary embodiment of a support catheter 400 comprising an elongate member 401 including a proximal end 402, a distal end 403, and a lumen 404 extending therethrough. The elongate member 401 may be fabricated from materials known in the art including, but not limited to, stainless steel, platinum, nitinol, Pebax, nylon, Delrin, polymethymethacrylate, polyurethane, polyimide, PTFE, combinations thereof, and the like. The lumen 404 further comprises a proximal section 404′ and a distal section 404″ wherein the diameter of proximal section 404′ is greater than that of distal section 404″.

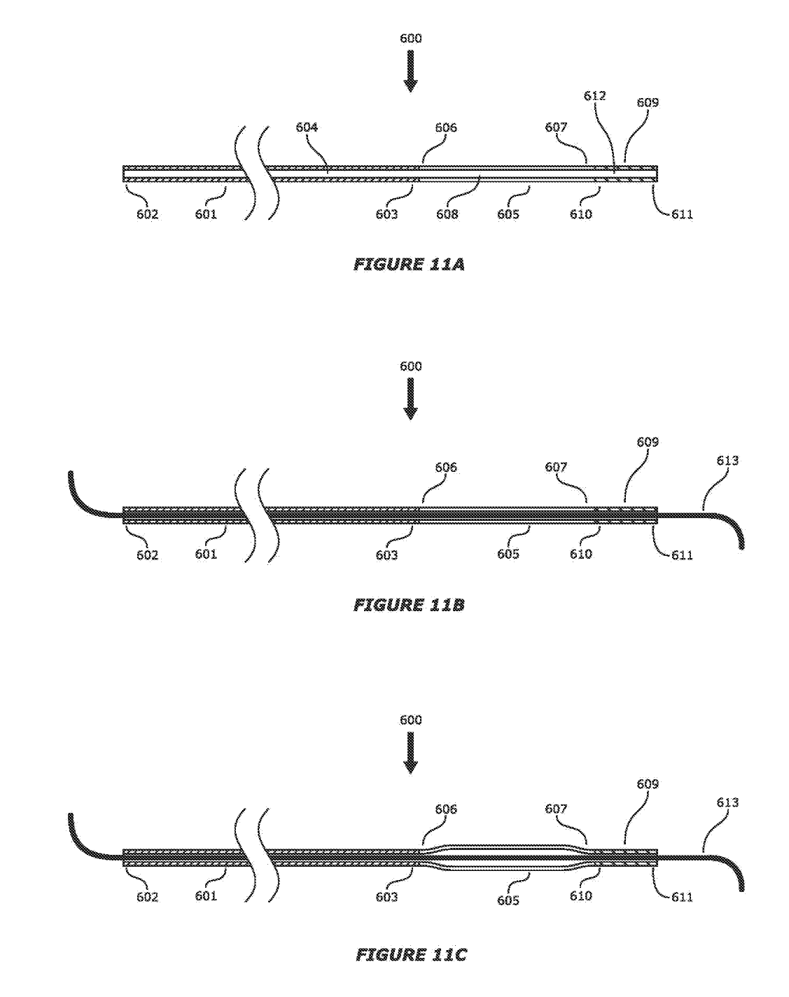

FIGS. 10A and 10B depict another exemplary embodiment of a support catheter 500 comprising a first elongate member 501 including a proximal end 502, a distal end 503, a lumen 504 extending therethrough, a window 510, and a tapped hole 511. The first elongate member 501 may be fabricated from materials known in the art including, but not limited to, stainless steel, platinum, nitinol, Pebax, nylon, Delrin, polymethymethacrylate, polyurethane, polyimide, PTFE, combinations thereof, and the like. The support catheter 500 further comprises a second elongate member 505 coaxially disposed within the lumen 504 of the first elongate member 501. The second elongate member 505 comprises a proximal end 506, a distal end 507, a lumen 508 extending therethrough, and a post 512 extending through window 510. The second elongate member 505 may be fabricated from materials known in the art including, but not limited to, stainless steel, platinum, nitinol, Pebax, nylon, Delrin, polymethymethacrylate, polyurethane, polyimide, PTFE, combinations thereof, and the like.

The support catheter 500 further comprises a threaded screw 509. The threaded screw 509 may be fabricated from materials known in the art including, but not limited to, stainless steel, aluminum, nylon, titanium, Delrin, polymethylmethacrylate, PTFE, combinations thereof, and the like. FIG. 10A shows the second elongate member 505 proximally retracted within the first elongate member 501, with the post 512 positioned at the proximal edge of the window 510. FIG. 10B shows the second elongate member 505 distally advanced out of the first elongate member 501, with the post 512 positioned at the distal edge of the window 510 and the distal end 507 of the second elongate member 505 extending distally beyond the distal end 503 of the first elongate member 501. The threaded screw 509 may be tightened to fix the first elongate member 501 and the second elongate member 505 relative to each other in the configurations shown in FIGS. 10A and 10B, or at any point in between.

FIGS. 11A-11C depict plan views of another exemplary embodiment of a support catheter 600. The support catheter 600 includes a first elongate member 601 with a proximal end 602, a distal end 603, and a lumen 604 extending therethrough. The first elongate member 601 may be fabricated from materials known in the art including, but not limited to, stainless steel, platinum, nitinol, Pebax, nylon, Delrin, polymethymethacrylate, polyurethane, polyimide, PTFE, combinations thereof, and the like.

The support catheter 600 further comprises an expandable segment 605. The expandable segment 605 is an elongate member that comprises a proximal end 606, a distal end 607, and a lumen therethrough 608. The expandable member 605 may be formed from materials known in the art including, but not limited to, polyurethane, polyethylene, PET, nylon, combinations thereof, and the like. Furthermore, the expandable segment 605 may be reinforced with additional materials such as braids, meshes, mandrels, liners, and the like, and/or may be fabricated from materials known in the art including, but not limited to nitinol, stainless steel, titanium, combinations thereof, and the like.

The expandable segment 605 may increase in diameter and/or cross sectional area when the pressure inside the support catheter 600 increases above a baseline level, and may optionally return to its initial diameter and/or cross sectional area when the pressure inside the support catheter 600 returns to baseline. The proximal end 606 of the expandable segment 605 is joined to the distal end 603 end of the first elongate member 601 such that the lumen 604 of the first elongate member 601 and the lumen 608 of the expandable segment 605 are in communication with each other using methods known in the art including, but not limited to, adhesive bonding, crimping, ultrasonic welding, combinations thereof, and the like.

The support catheter 600 further comprises a distal segment 609. The distal segment 609 comprises a proximal end 610, a distal end 611, and a lumen therethrough 612, and is fabricated from materials known in the art including, but not limited to, stainless steel, platinum, nitinol, Pebax, nylon, Delrin, polymethymethacrylate, polyurethane, polyimide, PTFE, combinations thereof, and the like. The distal segment 609 is relatively non-compliant and/or non-expandable. The proximal end 610 of the distal segment 609 is joined to the distal end 607 end of the expandable segment 605 such that the lumen 608 of the expandable segment 605 and the lumen 612 of the distal segment 609 are in communication with each other using methods known in the art including, but not limited to, adhesive bonding, crimping, ultrasonic welding, combinations thereof, and the like.

The lumens of the first elongate member 604, expandable segment 608, and distal segment 612 are sized to accept a guide wire. The outer diameter of the guide wire may comprise dimensions including, but not limited to not more than about 0.25 mm (0.010)″, 0.35 mm (0.014″), 0.45 mm (0.018″), 0.88 mm (0.035″), and the like. FIG. 11A depicts a plan view of the support catheter 600 with the expandable segment 605 in a collapsed state. FIG. 1 lB depicts a plan view of the support catheter 600 loaded over a guide wire 613. Furthermore, the lumens of the first elongate member 604 and expandable segment 608 may optionally be sized to allow fluid flow between the guide wire 613 and the annular wall of the first elongate member 601 and between the guide wire 613 and the annular wall of the expandable segment 605. FIG. 11C depicts a plan view of the support catheter 600 loaded over a guide wire 613 with the expandable segment 605 in the expanded or active state. Optionally (not shown), the first elongate member 601 may further comprise one or more exit ports that communicate between the lumen of the first elongate member 604 and the environment external to the support catheter 600. Furthermore, the first elongate member may include one or more features to reversibly seal the optional exit ports. The distal segment 609 may further comprise a gasket, o-ring, valve, or other features (not shown) for creating a seal between the annular wall of the distal segment lumen (612) and the guide wire 613.

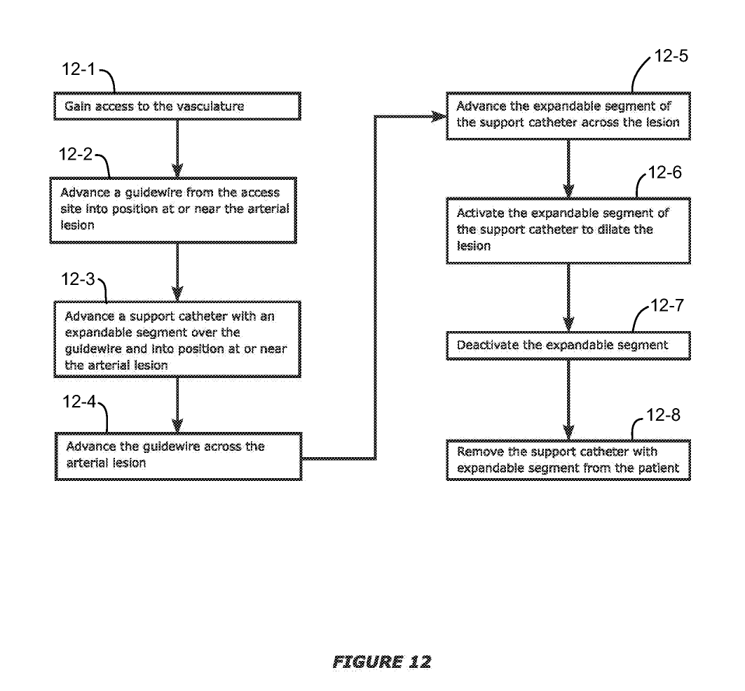

FIG. 12 depicts a flowchart describing an embodiment of a method for using a support catheter, such as any of those described herein, to dilate an arterial lesion prior to placing a treatment catheter (e.g., a balloon catheter, artherectomy catheter, etc., not shown) across or within the lesion. At Step 12-1, access to the patient's vasculature is obtained and, at Step 12-2, a guide wire is advanced into position at or near the arterial lesion. At Step 12-3, a support catheter is advanced over the guide wire into position at or near the arterial lesion. At Step 12-4, the guide wire is then placed across the arterial lesion and, at Step 12-5, the expandable segment of the support catheter is placed across and/or within the arterial lesion.

At Step 12-6, the expandable segment of the support catheter is activated, dilating the lesion and creating a larger bore for subsequent placement of a treatment device (e.g., a balloon catheter, an artherectomy catheter, etc.) than the passage created by the guide wire alone. At Step 12-7, the expandable segment of the support catheter is then deactivated. The process of advancing the support catheter across the lesion, activating the expandable segment of the support catheter, and deactivating the support catheter may be repeated if the lesion is longer than the expandable segment of the balloon catheter or if the lesion is resistant to complete crossing in a single advancement of the support catheter. Once the full length of the lesion has been dilated, at Step 12-8, the support catheter is removed from the patient. At this point, the treatment device may be advanced along the guide wire and into position across the lesion, and treatment of the lesion may proceed following standard clinical practice.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A catheter for treating a body lumen, comprising;
an elongate member comprising a proximal end, a distal end sized for introduction into a body lumen, a hub on the proximal end, and a fluid delivery lumen communicating between an inlet port on the hub and a transit port on a distal portion of the elongate member; and
an inflatable infusion element on the distal portion such that an interior of the inflatable infusion element communicates with the fluid delivery lumen via the transit port, an infusion exit port on a proximal side of the inflatable infusion element configured to deliver fluid introduced into the interior of the inflatable infusion element out the infusion exit port towards the proximal end.

2. The catheter of claim 1, wherein the elongate member comprises a plurality of transit ports on the distal portion communicating between the fluid delivery lumen and the interior of the inflatable infusion element.

3. The catheter of claim 1, wherein the inflatable infusion element comprises a plurality of exit ports on the proximal side thereof for delivering fluid from the interior of the inflatable infusion element.

4. The catheter of claim 1, further comprising a guide wire lumen extending from a guidewire port on the hub to an outlet on a distal tip of the elongate member distally beyond the inflatable infusion element.

5. The catheter of claim 1, wherein the inflatable infusion element comprises a balloon.

6. The catheter of claim 1, wherein the inflatable infusion element comprises compliant material.

7. The catheter of claim 6, wherein the inflatable infusion element comprises material configured to cause the exit port to open when fluid within the interior of the inflatable infusion element exceeds a predetermined pressure.

8. A catheter for treating a body lumen, comprising;
an elongate member comprising a proximal end, a distal end sized for introduction into a body lumen, a hub on the proximal end, and a fluid delivery lumen communicating between an inlet port on the hub and a transit port on a distal portion of the elongate member;
an inflatable infusion element comprising a proximal end attached to the elongate member adjacent the transit port such that the infusion element proximal end communicates with the fluid delivery lumen, and a distal end attached to the elongate member distal end; and
a tubular member comprising a cover or shield positioned over the transit port, the tubular member configured to at least partially open to provide an infusion exit port when fluid delivered into the fluid delivery lumen exceeds a predetermined pressure to deliver fluid through the transit port and out the infusion exit port towards the elongate member proximal end.

9. The catheter of claim 8, wherein the elongate member comprises a plurality of transit ports on the distal portion covered by the compliant tubular member.

10. The catheter of claim 8, wherein the compliant tubular member comprises a proximal end that is attached to an outer wall of the elongate member at multiple attachment locations around a circumference of the outer wall such that regions of the compliant tubular member between the attachment locations open to define a plurality of exit ports when the fluid in the fluid delivery lumen exceeds the predetermined pressure.

11. The catheter of claim 8, wherein the tubular member comprises an extension of a proximal leg of the inflatable infusion element.

12. The catheter of claim 11, wherein the proximal leg extension is attached to an outer wall of the elongate member at multiple attachment locations around a circumference of the outer wall such that regions of the proximal leg extensions between the attachment locations open to define a plurality of exit ports when the fluid in the fluid delivery lumen exceeds the predetermined pressure.

13. The catheter of claim 11, wherein the proximal leg comprises compliant material.

14. The catheter of claim 11, wherein the proximal leg comprises material that is biased against an outer wall of the elongate member to substantially seal the transit port, the material resiliently expandable when exposed to fluid within the infusion lumen above a predetermined threshold to open the transit port to deliver fluid from the fluid delivery lumen.

15. The catheter of claim 11, wherein the inflatable infusion element comprises a balloon.

16. The catheter of claim 11, wherein the inflatable infusion element comprises compliant material.

17. The catheter of claim 8, wherein the tubular member comprises compliant material.

18. The catheter of claim 8, wherein the tubular member comprises material that is biased against an outer wall of the elongate member to substantially seal the transit port, the material resiliently expandable when exposed to fluid within the infusion lumen above a predetermined threshold to open the transit port and deliver fluid from the fluid delivery lumen.

19. The catheter of claim 8, wherein the tubular member comprises a sleeve including a distal end attached to the elongate member to provide a substantially fluid tight seal, and a proximal end surrounding the elongate member.

20-26. (canceled)

27. A method for delivering fluids into a lumen or conduit, comprising:
introducing a distal end of a catheter into a lumen or conduit with an inflatable infusion element on the distal end in a collapsed condition;
expanding the inflatable infusion element to an expanded condition to partially or totally occlude the lumen or conduit; and
infusing fluid from the catheter into the lumen or conduit proximally relative to the inflatable infusion element while the inflatable infusion element remains in the expanded condition.

28-39. (canceled)
